(19) United States
(12) Patent Application Publication
(10) Pub. No.: US 2006/0034848 A1
(43) Pub. Date: Feb. 16, 2006

(54) Methods and compositions for treating Alzheimer's disease

(76) Inventors: Ayae Kinoshita, Sakyo-ku (JP); Christine A. F. Von Arnim, Kirchheim (DE); Dudley Strickland, Brookeville, MD (US); Bradley T. Hyman, Charlestown, MA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/983,976

(22) Filed: Nov. 8, 2004

Related U.S. Application Data
(60) Provisional application No. 60/518,272, filed on Nov. 7, 2003.

(51) Int. Cl. A61K 39/395; A61K 38/17; G01N 33/567

(52) U.S. Cl. 424/146.1; 435/7.2; 514/12

(57) ABSTRACT

The present invention relates to the treatment, diagnosis, and prophylactic prevention of Alzheimer's disease. More specifically, the present invention relates to methods and compositions for reducing or preventing the interaction of β-secretase (BACE) with low density lipoprotein receptor related protein (LRP).

### RELATED APPLICATIONS

This application claims the benefit under 35 U.S.C. § 119(e) of U.S. provisional application Ser. No. 60/518,272, filed Nov. 7, 2003, the disclosure of which is incorporated by reference herein.

### GOVERNMENT SUPPORT

This work was funded in part by the National Institutes of Health under grant numbers NIH AG12406, AG15379, P50AG05134, HL50784 and HL54710. The government may have certain rights in this invention.

### FIELD OF THE INVENTION

The present invention relates to the treatment, diagnosis, and prophylactic prevention of Alzheimer's disease. More specifically, the present invention relates to methods and compositions for reducing or preventing the interaction of β-secretase (BACE) with low density lipoprotein receptor related protein (LRP).

### BACKGROUND OF THE INVENTION

Alzheimer's disease (AD) is a late onset neurodegenerative disorder characterized by the extracellular deposition of insoluble aggregates composed of the 40 to 42 amino acid Aβ peptide in the brain (Glenner and Wong, Biochem. Biophys. Res. Commun. 120:885-890 (1984); Masters et al., EMBO J. 4:2757-2763 (1985)). Aβ peptide is derived from an integral membrane protein termed amyloid β-protein precursor protein (APP) (Tanzi et al., Science 235:880 (1987); Kang et al., Nature 325:733-736 (1987)). The function and metabolism of APP have been the subject of intensive study due to the fact that mutations in APP are associated with an autosomal dominant form of AD, (Goate et al., Nature 349:704-707 (1991)) and over-production of APP is the presumptive cause of AD in trisomy 21 (Tanzi et al., Science 235:880 (1987); Hyman et al., Proc. Natl. Acad. Sci. USA 92:3586-3590 (1995)). Multiple APP isoforms can be generated by alternatively splicing of mRNAs. The major isoforms in brain are APP695, APP751, and APP770 containing 695, 751 and 770 amino acids, respectively. These isoforms are transmembranous proteins having large extracellular regions, with hydrophobic membrane spanning domains and short cytoplasmic segments. APP is also a member of an evolutionary conserved family of proteins which include the APP-like proteins, APLP1 and APLP2 (Wasco et al., Proc. Natl. Acad. Sci. USA 89:10758-10762 (1992); Wasco et al., Nature Genet. 5:95-100 (1993); Slunt et al., J. Biol. Chem. 269:2637-2644 (1994)).

Secreted forms of APP are generated by proteolytic cleavages within their extracellular domain close to the transmembrane region. The extracellular regions of APP751, APP770, and APLP2 each contain a Kunitz protease inhibitor (KPI) domain encoded by an alternatively-transcribed exon (Kitaguchi et al., Nature 331:530-532 (1988); Tanzi et al., Nature 331:528-530 (1988); Wasco et al, Nature Genet. 5:95-100 (1993); Slunt et al., J. Biol. Chem. 269:2637-2644 (1994)). Secreted forms of APP having the KPI domain correspond to a protease inhibitor that has been identified separately and named protease nexin II (APP/PN-2) (Van Nostrand and Cunningham, J. Biol. Chem. 262:8508-8514 (1987); Oltersdorf et al., Nature 341:144-147 (1989); Van Nostrand et al., Nature 341:546-549 (1989)), a potent inhibitor of the blood coagulation factors IXa (Schmaier et al., J. Clin. Invest. 92:2540-2545 (1993)) and XIa (Van Nostrand et al., J. Biol. Chem. 265:9591-9594 (1990)). APP/PN-2 binds with high affinity to cultured fibroblasts (Johnson-Wood et al., Biochem. Biophys. Res. Commun. 200:1685-1692 (1994)), and APP/PN-2:proteinase complexes are internalized and degraded by cultured cells (Knauer and Cunningham, Proc. Natl. Acad. Sci. USA 79:2310-2314 (1982); Knauer et al., J. Cell. Physiol. 117:385-396 (1983)) although the mechanism for this process is unknown. Recent studies have identified the low density lipoprotein receptor-related protein (LRP) as the receptor responsible for the catabolism of another Kunitz-type inhibitor, tissue factor pathway inhibitor (TFPI) (Warshawsky et al., Proc. Natl. Acad. Sci. USA 91:6664-6668 (1994)).

The exact mechanisms whereby APP undergoes endocytosis are not well understood. This occurs at least in part via the low-density lipoprotein receptor-related protein (LRP); LRP was recently identified as capable of binding and mediating the internalization and degradation of APP as well as its complexes with proteinases (U.S. Pat. No. 6,156,311).

An aspartyl proteinase with β-secretase activity that cleaves the amyloid precursor protein (APP), has recently been cloned and is referred to as BACE (beta-site of APP-cleaving enzyme) (Sinha et al., 1999; Vassar et al., 1999; Yan et al., 1999). BACE is a type I membrane-associated aspartic protease. Since the BACE-mediated APP cleavage product C99 is the immediate precursor of amyloid-β, BACE is now regarded as a therapeutic target for inhibitor drugs to prevent amyloid-β deposition.

BACE is cotranslationally modified by N-glycosylation and further matures by complex glycosylation as well as proteolytic removal of its prodomain by a furin-like protease (Bennett et al., 2000; Capell et al., 2000; Creemers et al., 2001). Metabolic pulse chase experiments reveal that after glycosylation, BACE is rapidly transported to the Golgi apparatus and distal secretory pathway (Creemers et al., 2001). Measurable amounts of BACE are present on the plasma membrane (Huse et al., 2000; Kinoshita et al., 2003b). BACE is internalized from the cell surface to early endosomes and cycling of the protein between the cell membrane and the endosomes has been reported (Huse et al., 2000). This process is dependent on a dileucine motif in the cytoplasmic tail of BACE.

After endocytosis, APP interacts with BACE and gamma secretase which cleave amyloid-β from APP. Amyloid-β is then released from cells in the extracellular space, where in Alzheimer's disease it aggregates as senile plaques.

Because catabolism of APP has been shown to generate the Aβ peptide, which is believed to be the causative agent of Alzheimer's Disease, there is a need for compositions and methods which reduce the interaction of APP with secretases and the subsequent catabolism of APP.

### SUMMARY OF THE INVENTION

Recently, we have shown that both APP and BACE are co-internalized from the cell surface to early endosomes (Kinoshita et al., 2003b). APP is known to interact at the cell surface and be internalized by LRP, a multifunctional endocytic receptor (Kounnas et al., 1995; Ulery et al., 2000; Rebeck, 2001; Pietrzik et al., 2002).

LRP is a type I integral membrane protein which has a large extracellular domain and a relatively small cytoplasmic tail, by which LRP interacts with multiple intracellular adaptor and scaffold proteins (Herz and Strickland, 2001; Li et al., 2001). LRP also interacts with multiple ligands, including APP, apolipoprotein E and α2-macroglobulin (Herz and Strickland, 2001; Li et al., 2001). We, therefore, investigated the possibility of an interaction between LRP and BACE.

Using both a fluorescence resonance energy transfer (FRET) based assay of protein proximity and co-immunoprecipitation, we now demonstrate that the light chain of LRP interacts with BACE, that the site of interaction is in the intracellular domain of LRP and appears to depend on the first NPxY (SEQ ID NO:3) motif of LRP.

According to one aspect of the invention, methods for modulating β-secretase activity in a subject are provided. The methods include contacting a mammalian cell with an agent that reduces the amount or rate of binding of β-secretase (BACE) with the low density lipoprotein receptor-related protein (LRP). In some embodiments, the agent is an agent which binds to the BACE protein, preferably an antibody or an antigen binding fragment thereof that binds to BACE protein, or a functional derivative or fragment of LRP that comprises a NPxY (SEQ ID NO:3) motif or an intracellular domain of LRP. In other embodiments, the agent is an agent that binds to LRP, preferably an antibody or an antigen binding fragment thereof that binds to LRP or a functional derivative or fragment of BACE that binds to LRP. In certain embodiments, the contacting occurs in vitro.

In another aspect of the invention, methods for treating or preventing Alzheimer's disease are provided. The methods include administering to an animal one or more agents that bind to the BACE-binding site on LRP (Group I agents) and/or one or more agents that bind to the LRP-binding site found on BACE (Group II agents) in an amount effective to reduce the rate of BACE binding to LRP.

In some embodiments, the agent is an agent which binds to the BACE protein, preferably an antibody or an antigen binding fragment thereof that binds to BACE protein or a functional derivative or fragment of LRP that comprises a NPxY (SEQ ID NO:3) motif or an intracellular domain of LRP. In other embodiments, the agent is an agent that binds to LRP, preferably an antibody or an antigen binding fragment thereof that binds to LRP or a functional derivative or fragment of BACE that binds to LRP. In preferred embodiments, the animal is a human.

According to a third aspect of the invention, pharmaceutical compositions are provided that include one or more agents that bind to the BACE-binding site of LRP (Group I agents) and/or one or more agents that bind to the LRP-binding site of BACE (Group II agents), and a pharmaceutically acceptable carrier. In some embodiments, the agent that binds to the BACE-binding site of LRP is an antibody or an antigen binding fragment thereof that binds to the BACE-binding site of LRP, or a functional derivative or fragment of BACE that binds to LRP. In other embodiments, the agent that binds to the LRP-binding site of BACE is an antibody or an antigen binding fragment thereof that binds to the LRP-binding site of BACE, or a functional derivative or fragment of LRP that comprises a NPxY (SEQ ID NO:3) motif or an intracellular domain of LRP.

In a forth aspect of the invention, methods for identifying compounds that modulate the interaction of LRP and BACE are provided. The methods include providing a reaction mixture that comprises LRP protein and/or a fragment thereof that includes the cytoplasmic tail including the first NPXY (SEQ ID NO:3) motif, and BACE protein or a fragment thereof that binds specifically to LRP, contacting the reaction mixture with a test compound, determining a level of interaction of LRP or fragment thereof with BACE in the absence and in the presence of the test compound, and comparing the level of interaction of LRP or fragment thereof with BACE in the absence and in the presence of the test compound. A test compound that modulates the interaction relative to the level of interaction in the absence of the test compound is a compound that modulates the interaction of LRP with BACE. In some embodiments, the LRP protein and/or a fragment thereof further includes the extracellular domain.

In certain embodiments, the test compound is a small molecule. In other embodiments, the test compound is an antibody that binds to LRP or BACE, or an antigen-binding fragment thereof. In still other embodiments, the test compound is a fragment of LRP or a fragment of BACE.

According to yet another aspect of the invention, methods for identifying compounds that modulate the cleavage of APP by BACE are provided. The methods include providing a reaction mixture that comprises LRP protein and/or a fragment thereof that includes the cytoplasmic tail including the first NPXY (SEQ ID NO:3) motif or an intracellular domain of LRP, BACE protein, and APP, contacting the reaction mixture with a test compound, determining a level of cleavage of APP by BACE in the absence and in the presence of the test compound, and comparing the cleavage in the absence and in the presence of the test compound. A test compound that modulates cleavage of APP relative to the level of cleavage in the absence of the test compound is a compound that modulates APP cleavage. In some embodiments, the LRP protein and/or a fragment thereof further includes the extracellular domain.

In certain embodiments, the test compound is a small molecule. In other embodiments, the test compound is an antibody that binds to LRP or BACE, or an antigen-binding fragment thereof. In still other embodiments, the test compound is a fragment of LRP or a fragment of BACE.

According to a further aspect of the invention, methods for modulating LRP signaling activity in a subject are provided. The methods include contacting a mammalian cell with an agent that modulates the amount or rate of binding of β-secretase (BACE) with the low density lipoprotein receptor-related protein (LRP), thereby modulating the cleavage of LRP by BACE. Modulation can be increasing or decreasing the amount or rate of binding. The contact can occur in vitro.

In some embodiments, the agent is an agent which binds to the BACE protein. Preferably the agent is an antibody or an antigen binding fragment thereof that binds to BACE protein, or a functional derivative or fragment of LRP that comprises a NPxY (SEQ ID NO:3) motif, or a functional derivative or fragment of LRP that comprises an intracellular domain of LRP.

In other embodiments, the agent is an agent that binds to LRP. Preferably the agent is an antibody or an antigen binding fragment thereof that binds to LRP, or a functional derivative or fragment of BACE that binds to LRP. In certain embodiments, the agent binds to an intracellular domain of LRP.

Use of the foregoing compounds and agents in the preparation of medicaments is also provided, particularly for use in treatment of Alzheimer's disease.

Other aspects, embodiments features and advantages of the present invention will be set forth in the detailed description of preferred embodiments that follows, and in part will be apparent from the description or may be learned by practice of the invention.

### BRIEF DESCRIPTION OF THE DRAWING

FIG. 1 shows localization of BACE and LC in transfected H4 cells. FIG. 1A: BACE-V5 and LC-myc co-transfected H4 cells were immunostained with anti-V5 (mouse, visualized by Cy5) and anti-myc (rabbit, visualized by Cy3), then washed thoroughly, and FITC-conjugated anti-EEA was applied. LC (shown in red pseudocolor) and EEA (blue) showed a complete match, suggesting that LC is mainly localized in endosomal compartment, where BACE (green) is colocalized with LC. FIG. 1B: BACE-GFP (shown in green) and LC-myc (red) were co-transfected and then immunostained with anti-myc (rabbit, visualized by Cy3) and anti-GM130 (mouse, visualized by Cy5, shown in blue). As seen in the superimposed figure, BACE is localized in both secretory pathway and endosomal compartment, however, LC is not co-localized with Golgi marker, GM130.

FIG. 2 depicts co-immunoprecipitation of LC with BACE. FIG. 2A: Extracts from wild type LC-myc (wt) and BACE-V5 transfected H4 cells or NPxY (SEQ ID NO:3) double mutant LC-myc (mut) and BACE-V5 transfected H4 cells were immunoprecipitated with mouse anti-V5 antibody and probed with rabbit anti-myc antibody. LC is recognized as approximately 100 kDa band in our experiment as seen in the lysate lane. A specific band of the same size was found only in the lane 1 from the lysates of the wild type LC-myc expressing cells. Cell extract proteins from either transfected H4 cells that were not immunoprecipitated with anti-V5 antibody (lane2) or anti-V5 antibody treatment without transfected cell lysates (lane3) did not show the immunoreactive band. The mutant LC-myc was not immunprecipitated by anti-V5 antibody. FIG. 2B: Immunoprecipitates of LC-myc were probed with an anti-BACE antibody, showing immunoreactive bands for BACE (65-90 kDa) only in lane 1, as well as in the lysate lane.

FIG. 3 shows FLIM analysis of the proximity between LC and BACE molecules within the cells. H4 cells were co-transfected with LC-myc (labeled by Cy3) and BACE-V5 (labeled by FITC) for the analysis (FIG. 3A). For the negative control, primary antibody (anti-myc antibody) for the acceptor fluorophore was not applied (FIG. 3B). The intensity image shows the standard immunostaining pattern for BACE. Color coded FLIM image shows the lifetimes (picosecond) of FITC in the presence of donor Cy3. The lifetime reflects the proximity between LC and BACE, demonstrated as pseudocolor in red-yellow-green-blue. Colorimetric scale shows fluorescence lifetime in picoseconds. FLIM image shows that there is a close proximity between LC and BACE mostly in the endosomal compartments (FIG. 3A). The negative control showed a homogenous image demonstrated by blue-green pseudocolor, showing a single lifetime of FITC.

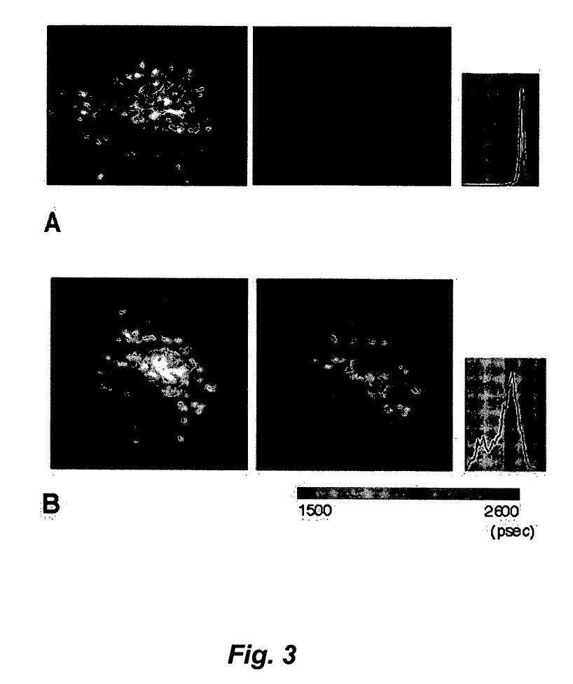

FIG. 4 shows the reagents used in Example 2. Shown are the constructs and antibodies used in this study.

FIG. 5 depicts the localization of BACE and mLRP1 in transfected H4 cells. FIG. 5A: BACE-V5 (green) and mLRP1-myc (red) co-transfected H4 cells were immunostained with anti-V5 mAb (visualized by Cy5) and rabbit anti-myc Ab (visualized by Cy3) followed by a FITC conjugated antibody to the endosomal marker EEA1 (shown in blue). FIG. 5B: BACE-GFP (green) and mLRP1-myc (red) were co-transfected and then immunostained with rabbit anti-myc (visualized by Cy3) and an antibody to the Golgi marker GM130 (visualized by Cy5, shown in blue). FIG. 5C: To demonstrate cell surface localization, BACE-V5 (blue) and myc-(N-terminus)-LC (green) were co-transfected and then immunostained with anti-myc mAb (visualized by FITC) and rabbit anti-BACE-NT Ab (visualized by Cy5) without permeabilizing the cell membrane. Alexa-555-labeled Cholera Toxin B (CTx-B), used to visualize lipid rafts, was added for 20 min after thoroughly washing the primary and before adding the secondary Ab.

FIG. 6 shows co-immunoprecipitation of LC/LRP with BACE. FIG. 6A: Extracts from mLRP1-myc and BACE-V5 transfected H4 cells were immunoprecipitated with mouse anti-V5 Ab and probed with rabbit antimyc Ab. Anti-myc recognizes precursor endoplasmatic reticulum and Golgi forms (labeled p) as well as β-chains of mature proteins (labeled b) as described before (positive control, lane 5, (Mikhailenko et al., 2001). Specific bands of all isoforms were found in pull-down (lane 1) from lysates of mLRP1-myc and BACE-V5 expressing cells. FIG. 6B: Immunoprecipitates of mLRP1-myc were probed with an anti-BACE Ab, showing immunoreactive bands for BACE (app. 60 kDa and 75 kDa) in lane1. The 60 kDa band represents endogenous BACE and the 75 kDa band transfected BACE-V5 (lane 5). FIG. 6C: Human brain extracts were immunoprecipitated with rabbit anti-BACE-CT Ab and probed with 11H4, a mAb to the LRP cytoplasmic domain. LC is recognized as 85 kDa band in lane 5 (positive control, human brain tissue). A specific band of the same size was found after co-immunoprecipitation with anti-BACE-Ab (lane 1). Identical results were observed when probing with 5A6, another LC-specific Ab (data not shown). Negative controls as described above are shown in lanes 2 and 3. Supernatants are shown in lane 4.

FIG. 7 depicts FLIM analysis of the proximity between LRP and BACE within cells. H4 cells were cotransfected with mLRP1-myc (FIG. 7A, unlabeled; FIG. 7B, labeled by Cy3) and BACE-V5 (labeled by FITC). N2a cells were stained for endogenous LRP with 1H4 (labeled by Cy3) and Anti-BACE CT Ab (labeled by Alexa488) for the analysis (FIG. 7D) or only with the donor fluorophore in the negative control (FIG. 7C). Primary neurons were stained for endogenous LRP with 11H4 (Cy3) and Anti-BACE CT Ab (FITC) for the analysis (FIG. 7F) or only with the donor fluorophore (FIG. 7E). The intensity images show the standard immunostaining pattern for BACE. The color-coded FLIM image shows the lifetimes (ps) of FITC in the presence of donor Cy3.

FIG. 8 shows FLIM analysis of LC and BACE proximity on cell surface. H4 cells were co-transfected with myc-LC (labeled by FITC) and BACE-V5 (FIG. 8A, unlabeled; FIGS. 8B, 8C, labeled by Cy3). Cholesterol depletion was performed with lovastatin/mevalonate for 24 h and MPCD for 10 min (FIG. 8C). The intensity image shows a typical immunostaining pattern for surface LRP. The color-coded FLIM image shows the lifetimes (ps) of FITC in the absence (FIG. 8A) or presence of the acceptor Cy3 (FIGS. 8B,8C). The shorter FITC lifetimes, represented by red-yellow pseudocolor, appear in distinct spots on the cell surface (FIG. 8B) and can be abolished by cholesterol depletion (FIG. 8C).

FIG. 9 shows the results of a BACE internalization assay. Internalization of biotinylated myc-BACE was monitored over 40 min in LRP null CHO cells (13-5-1) cells co-transfected with either empty vector (peGFP ⋄) or LC-GFP (▪). No change of the basic endocytosis rate of BACE with LRP co-transfection was observed. Given are the means and S.D. after both transfection and measurement were carried out in triplicate representing three independent assays.

FIG. 10 shows the results of a LRP shedding assay. N2a cells were transfected with SEAP-LRP, β-gal and empty vector (Topo) or with a plasmid encoding WT-BACE or a catalytically inactive BACE mutant (BACE D93/289A). Shown is the alkaline phosphatase activity in the conditioned medium normalized to β-gal activity. Given are the means and S.D. after both transfection and measurement were carried out in triplicate representing three independent assays.

FIG. 11 depicts a LRP CTF western. N2a cells were cotransfected with LC-myc and either empty vector, BACE, or a catalytically inactive BACE mutant (deadBACE) and treated with vehicle or DAPT (12 h) treatment. An increase of CTF intensity was detected after DAPT treatment and BACE cotransfection. Shown is the intensity of a representative blot out four experiments without DAPT treatment and two with DAPT treatment.

FIG. 12 shows that BACE increases release of LRP-ICD. HEK 293 cells were transfected with LRPGal4/VP16 (May et al., 2002) in the absence or presence of BACE, and luciferase activity determined. Activity relative to beta galactosidase is shown; average of triplicate analyses. The addition of BACE led to a statistically highly significant increase in luciferase activity suggesting that LRP undergoes BACE cleavage, leading to subsequent release of the cytoplasmic domain and translocation to the nucleus to activate the Gal4 assay.

### DETAILED DESCRIPTION OF THE INVENTION

The current invention describes the interaction of low density lipoprotein receptor-related protein (LRP) with β-secretase (BACE). It follows that interfering with this interaction alters the colocalization of APP and BACE, which can reduce Aβ generation, and therefore is a therapeutic mechanism for Alzheimer's disease.

Agents that inhibit LRP/BACE binding and/or endocytosis are useful in accordance with the invention for reduction of Aβ production and treatment of Alzheimer's disease. These inhibitory agents can take at least two forms: (1) agents which bind to the BACE-binding site on the LRP protein (Group I agents) and agents which bind to the LRP-binding site found on BACE (Group II agents).

As used herein, an agent is said to reduce the amount or rate of binding if the amount or rate of binding is less in the presence of the agent than when the agent is absent. Under conditions when the amount or rate of reduction is nearly complete, there will be an actual inhibition or total blocking of binding.

As used herein, the agents of the present invention, i.e., the Group I agents and the Group II agents, may be any composition of matter provided that it has the ability to bind to the BACE-binding site on LRP (Group I) and/or the ability to bind to the LRP-binding site on BACE (Group II). Suitable agents exhibiting these properties include, but are not limited to, peptides, antibodies, carbohydrates, nucleic acids, vitamins, pharmaceutical agents, and the like, including derivatives thereof.

The agents of the present invention may be identified and/or prepared according to any of the methods and techniques known to those skilled in the art. These agents, particularly peptide agents and antibody agents, may occur or be produced as monomers, dimers, trimers, tetramers or multimers. Such multimers can be prepared using enzymatic or chemical treatment of the native receptor molecules or be prepared using recombinant techniques. Preferably, the agents of the present invention are selected and screened at random or rationally selected or designed using protein modeling techniques.

For random screening, candidate agents are selected at random and assayed for their ability to reduce selectively/specifically the amount or rate of binding of the β-secretase protein (BACE) to the LRP protein. Any of the suitable methods and techniques known to those skilled in the art may be employed to assay candidate agents.

For rational selection or design, the agent is selected based on the configuration of the LRP binding site found on BACE or the BACE binding site found on LRP. Any of the suitable methods and techniques known to those skilled in the art may be employed for rational selection or design. For example, one skilled in the art can readily adapt currently available procedures to generate antibodies, peptides, pharmaceutical agents and the like capable of binding to a specific peptide sequence of LRP or BACE, preferably the light chain of LRP, more preferably the NPxY (SEQ ID NO:3) sequence(s) of LRP that is required for BACE binding. Illustrative examples of such available procedures are described, for example, in Hurby et al., “Application of Synthetic Peptides: Antisense Peptides,” in Synthetic Peptides, A User's Guide, W. H. Freeman, N.Y., pp. 289-307 (1992); Kaspczak et al., Biochemistry 28:9230 (1989); and Harlow, Antibodies, Cold Spring Harbor Press, N.Y. (1990).

The agents of the present invention can alternatively be identified using modification of methods known in the art. For example, suitable peptide agents may be identified using the filter binding assay described by Mischak et al. (Mischak et al., J. Gen. Virol. 69:2653-2656 (1988) and Mischak et al., Virology 163:19-25 (1988)), wherein the peptide is applied to a suitable membrane, such as nitrocellulose, and the membrane is saturated with a detergent mixture in order to block any non-specific binding. The treated membrane is then incubated with labeled rhinovirus, e.g. with HRV2 labeled with ³⁵S-methionine, in order to check the specific binding. After washing and drying of the membrane, specific binding can be visualized by autoradiography.

Other methods include fluorescence energy transfer assays, as described in the Examples. Briefly, one can identify inhibitors of LRP/BACE interaction by observing differences in fluorescence energy transfer of the fluorescently labeled proteins in the presence and absence of potential agents.

As noted above, the Group I agents of the present invention include those agents which bind directly to the BACE-binding site on found on the LRP protein. Additionally, the Group I agents of the present invention bind to the LRP in interfering proximity with the BACE binding site or bind to LRP in such a manner so as to conformationally alter the BACE binding site. Suitable Group I agents can therefore be first identified by their ability to bind LRP and then by their ability to reduce the amount or rate at which BACE binds to LRP. Illustrative examples of Group I agents of the present invention include, but are not limited to: soluble fragments of BACE that bind to LRP; and anti-LRP antibodies or binding fragments thereof.

Preferred Group I agents are based on and derived from the amino acid sequence of the BACE protein. An especially preferred type of Group I agent is an isolated fragment of BACE, such as a soluble fragment of BACE which contains the LRP binding site, particularly one which binds selectively (i.e., specifically) to LRP and not to other members of the LDL receptor protein family. Such agents act as competitive inhibitors of BACE binding to its receptor in vitro as well as in vivo.

The preferred fragments of BACE are soluble under physiological conditions. The termini of these polypeptides can be shortened as desired, provided that the binding capacity for the LRP protein remains intact. The preferred amino acid sequence of BACE corresponds to the human protein. Suitable BACE sequences can also be derived from the amino acid sequence of BACE isolated from other mammals.

BACE, or a LRP-binding fragment thereof, may be produced using any of the methods and techniques known to those skilled in the art. For example, BACE can be purified from a source which naturally expresses the protein, can be isolated from a recombinant host which has been altered to express BACE or fragment thereof, or can be synthesized using protein synthesis techniques known in the art. The skilled artisan can readily adapt a variety of techniques in order to obtain Group I peptide agents which contain the LRP binding site found on BACE.

The isolation of native BACE proteins is known in the art. In order to generate fragments of BACE which contains the LRP binding site, isolated native protein may be converted by enzymatic and/or chemical cleavage to generate fragments of the whole protein, for example by reacting cell lines which express a BACE protein with an enzyme such as papain or trypsin or a chemical such as cyanogen bromide. Proteolytically active enzymes or chemicals are preferably selected in order to release the region that binds LRP. Fragments which contain the LRP binding site, especially fragments which are soluble under physiological conditions, can then be isolated using known methods.

Alternatively, BACE or a fragment of BACE may be expressed in recombinant bacteria or yeast, each of which is well known in the art.

The Group II agents of the present invention include compositions which bind to the LRP binding site found on BACE. Additionally, the Group II agents of the present invention include compositions that bind to BACE in interfering proximity to the LRP binding site.

Suitable Group II agents can therefore be first identified by their ability to bind to BACE and then by their ability to reduce the amount or rate at which BACE binds to LRP. Illustrative examples of the Group II agents of the present invention include, but are not limited to, antibodies which bind to the LRP binding site found on BACE and soluble fragments of LRP, particularly the light chain of LRP, preferably those fragments of LRP that include a NPxY (SEQ ID NO:3) sequence motif, or a peptide including this motif.

Fragments of BACE and LRP useful in accordance with the invention include BACE and LRP polypeptide fragments, which are BACE and LRP polypeptides that are not full-length polypeptides. For example, a fragment of LRP may have an amino acid sequence that is the sequence of LRP reduced in length at one and/or both ends by up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 525, 550, 575, 600, 625, 650, 675, 700, 725, 750, 800, 900, 1000 or more amino acids (including every integer therebetween), as long as the LRP fragment retains a functional capability of LRP as is contemplated herein, e.g., binding to BACE. The fragments may be identified as retaining a functional capability of full-length LRP polypeptide using the assays described herein.

In addition to wild-type (e.g., natural) polypeptides and fragments thereof that are useful in the invention, variant or modified polypeptides and/or fragments that are useful in the methods and composition of the invention can be identified by one of ordinary skill in the art. In some embodiments, the modified polypeptide is a modified LRP or BACE polypeptide or fragment thereof. As used herein the term “modified LRP polypeptide or fragment thereof” or a “modified BACE polypeptide or fragment thereof” means a LRP or BACE polypeptide that has one or more modifications in amino acid sequence from the amino acid sequence of a natural (wild-type) LRP or BACE polypeptide or fragment thereof. One of ordinary skill will understand that a modified LRP or BACE polypeptide or fragment thereof may have an addition, deletion, substitution, or alteration of amino acid sequence, or any combination of these types of amino acid changes. For example, an LRP or BACE polypeptide or fragment thereof that is useful in the claimed methods of the invention may be a LRP polypeptide or fragment that comprises a BACE binding site, but has a region other than its binding site sequence added to, deleted, or substituted with a sequence that is different than the natural (e.g. wild-type) sequence.

In addition, a modified LRP or BACE polypeptide or fragment may include a binding site, and also include one or more amino acids that are additional within (or at either or both ends of) the natural sequence of the LRP or BACE polypeptide or fragment thereof. In some embodiments of the invention, the modified LRP or BACE polypeptide is a fusion protein. One of ordinary skill in the art will understand how to prepare and test fusion proteins for use in the methods of the invention.

The skilled artisan will realize that some modified polypeptides of the invention may have conservative amino acid substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

It will be understood that one can make a conservative substitution to the amino acid sequence of a polypeptide of the invention and still have it be functional in the assays of the invention. For example, a conservative substitution can be made to the amino acid sequence of a LRP polypeptide or fragment thereof and still have the polypeptide retain its functional activity (e.g., specific BACE-binding characteristics), and thus its usefulness in the assay methods of the invention. Conservative amino-acid substitutions in an amino acid sequence can be made by alteration of a nucleic acid encoding the polypeptide. Such substitutions can be made by a variety of methods known to one of ordinary skill in the art. For example, amino acid substitutions may be made by PCR-directed mutation, site-directed mutagenesis (such as using the method of Kunkel, Proc. Nat. Acad. Sci. U.S.A. 82: 488-492, 1985 or various commercially-available methods), or by chemical synthesis of a gene encoding a BACE or LRP polypeptide. Where amino acid substitutions are made to a small fragment of a BACE or LRP polypeptide, the substitutions can be made by directly synthesizing the peptide. The activity of functionally equivalent fragments of BACE or LRP polypeptides can be tested by cloning the gene encoding the altered BACE or LRP polypeptide into a bacterial or mammalian expression vector, introducing the vector into an appropriate host cell, expressing the altered polypeptide, and testing for a functional capability of the BACE or LRP polypeptide in the assays of the invention. Peptides that are chemically synthesized can be tested directly for function, e.g., the ability to bind to BACE or LRP.

Preferred Group I and Group II agents include antibodies and antibody fragments which are capable of binding to a residue found on BACE or LRP and consequently act as a competitive inhibitor for LRP or BACE binding, respectively. The most preferred antibodies or antibody fragments of the present invention bind to a LRP-specific epitope in BACE or a BACE-specific epitope in LRP. Antibodies, or other Group I or Group II agents such as peptides containing the amino acid sequence NPxY (SEQ ID NO:3), which bind to epitopes within this sequence reduce the amount or rate of BACE binding to LRP.

The antibodies of the present invention include polyclonal and monoclonal antibodies, as well as antibody fragments and derivatives that contain the relevant antigen binding domain of the antibodies. Such antibodies or antibody fragments are preferably used in the diagnostic and therapeutic embodiments of the present invention.

Suitable monoclonal and polyclonal antibodies may be prepared by any of the methods and techniques well known in the art, such as described in, for example, A. M. Campbell, Monoclonal Antibody Technology: Laboratory Techniques in Biochemistry and Molecular Biology, Elsevier Science Publishers, Amsterdam, The Netherlands (1984) and Harlow; Antibodies, Cold Spring Harbor Press, N.Y. (1989). For example, an antibody capable of binding to a domain of BACE or LRP can be generated by immunizing an animal with a polypeptide whose sequence is encoded by that domain. Any animal (mouse, rabbit, etc.) which is known to produce antibodies can be utilized to produce antibodies with the desired specificity and suitable methods for immunization of these animals are well known in the art, including, for example, subcutaneous or intraperitoneal injection of the polypeptide. One skilled in the art will recognize that the amount of polypeptide used for immunization will vary based on a number of factors, including the animal which is immunized, the antigenicity of the polypeptide selected, and the site of injection.

The polypeptides used as an immunogen may be modified as appropriate or administered in an adjuvant in order to increase the peptide antigenicity. Suitable methods increasing antigenicity are well known in the art, and include, for example, coupling the antigen with a heterologous protein (such as globulin or β-galactosidase) or through the inclusion of an adjuvant during immunization.

A preferred method of generating monoclonal antibodies comprises removing spleen cells from the immunized animals, fusing these cells with myeloma cells, such as SP2/0-Ag14 myeloma cells, and allowing them to become monoclonal antibody-producing hybridoma cells. Any one of a number of methods well known in the art may be used to identify the hybridoma cell which produces an antibody with the desired characteristics. These include screening the hybridomas with an ELISA assay, western blot analysis, or radioimmunoassay (Lutz et al., Exp. Cell Res. 175:109-124 (1988); Kishimoto et al., Proc. Natl. Acad. Sci USA 87:2244-2248 (1990)). Hybridomas secreting the desired antibodies are cloned and the class and subclass of the secreted antibodies are determined using procedures known in the art (Campbell, A. M., Monoclonal Antibody Technology: Laboratory Techniques in Biochemistry and Molecular Biology, Elsevier Science Publishers, Amsterdam, The Netherlands (1984)).

For polyclonal antibodies, antibody-containing antisera is preferably isolated from the immunized animal and is screened for the presence of antibodies with the desired specificity using one of the above-described procedures.

The present invention further provides hybrid cell lines which secrete monoclonal antibodies selective for the Group I agents and/or Group II agents. These monoclonal antibodies are capable of wholly or partially neutralizing the activity of the polypeptides or specifically binding to one of the said polypeptides. These monoclonal antibodies can be used for qualitative and/or quantitative measurement or for purification of the polypeptides according to the invention. The present invention therefore also includes test systems which contain the monoclonal antibodies herein described.

Antibodies may be used as an isolated whole antibody, or can be used as a source for generating antibody fragments which contain the antigen binding site of the antibody. Examples of such antibody fragments include, but are not limited to the Fv, the F(ab), the F(ab)₂, fragment, as well as single chain antibodies.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

Various methods known in the art can be used to generate such fragments without undue experimentation. Recombinant techniques are preferred for generating large quantities of antibodies, antibody fragments and single chain antibodies, as described, for example, in Pluckthum, Bio/Technology 10:163-167 (1992); Carter et al., Bio/Technology 10:167-170 (1992); and Mullinax et al., Biotechniques 12:864-869 (1992). In addition, recombinant techniques may be used to generate heterobifunctional antibodies.

In general, recombinant production of antibodies, antibody fragments or derivatives thereof, uses mRNA encoding an antibody which is isolated from hybridoma cells that produce the desired antibody. This mRNA is then used as a source for generating a cDNA molecule which encodes the antibody, or a fragment thereof. Once obtained, the cDNA may be amplified and expressed according to known methods in a variety of eukaryotic and prokaryotic hosts.

The present invention further includes derivatives of antibodies (antibody derivatives). As used herein, an “antibody derivatives” contain an antibody of the present invention, or a fragment thereof, as well as an additional moiety which is not normally a part of the antibody. Such moieties may improve the solubility, absorption, biological half-life, etc., of the antibody, decrease the toxicity of the antibody, eliminate or attenuate any undesirable side effect of the antibody, or serve as a detectable marker of the presence of the antibody. Moieties capable of mediating such effects are well known in the art.

Detectably labeled antibodies constitute a special class of the antibody derivatives of the present invention. An antibody is said to be “detectably labeled” if the antibody, or fragment thereof, is attached to a molecule which is capable of identification, visualization, or localization using known methods. Suitable detectable labels include radioisotopic labels, enzyme labels, non-radioactive isotopic labels, fluorescent labels, toxin labels, affinity labels, chemiluminescent labels and nuclear magnetic resonance contrast agents.

Illustrative examples of suitable enzyme labels include, but are not limited to, luciferase, malate dehydrogenase, staphylococcal nuclease, delta-5-steroid isomerase, yeast-alcohol dehydrogenase, alpha-glycerol phosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include, but are not limited to, ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd etc. ¹¹¹In is a preferred isotope where in vivo imaging is used since its avoids the problem of dehalogenation of the ¹²⁵I or ¹³¹I-labeled monoclonal antibody by the liver. In addition, this radionucleotide has a more favorable gamma emission energy for imaging (Perkins et al., Eur. J. Nucl. Med 10:296-301 (1985); Carasquillo et al., J. Nucl. Med. 28:281-287 (1987)). For example, ¹¹¹In coupled to monoclonal antibodies with 1-(p-isothiocyanatobenzyl)-DPTA has shown little uptake in non-tumorous tissues, particularly the liver, and therefore enhances specificity of tumor localization (Esteban et al., J. Nucl. Med. 28:861-870 (1987)).

Illustrative examples of suitable non-radioactive isotopic labels include, but are not limited to, ¹⁵⁷Gd, ⁵⁵Mn, ⁶²Dy, ⁵²Tr, and ⁵⁶Fe.

Illustrative examples of suitable fluorescent labels include, but are not limited to, an ¹⁵²Eu label, a fluorescent protein (including green fluorescent protein (GFP), enhanced green fluorescent protein (EGFP) and yellow fluorescent protein (YFP)), a fluorescein label, an isothiocyanate label, a rhodamine label, a phycoerythrin label, a phycocyanin label, an allophycocyanin label, an o-phthaldehyde label, and a fluorescamine label.

Suitable chemical toxins or chemotherapeutic agents include members of the enediyne family of molecules, such as calicheamicin and esperamicin. Chemical toxins can also be taken from the group consisting of duocarmycin (see e.g., U.S. Pat. No. 5,703,080 and U.S. Pat. No. 4,923,990), methotrexate, doxorubicin, melphalan, chlorambucil, ARA-C, vindesine, mitomycin C, cis-platinum, etoposide, bleomycin and 5-fluorouracil. Toxins that are less preferred in the compositions and methods of the invention include poisonous lectins, plant toxins such as ricin, abrin, modeccin, botulinum and diphtheria toxins. Of course, combinations of the various toxins could also be coupled to one antibody molecule thereby accommodating variable cytotoxicity. Other chemotherapeutic agents are known to those skilled in the art.

Illustrative examples of chemiluminescent labels include a luminal label, an isoluminal label, an aromatic acridinium ester label, an imidazole label, an acridinium salt label, an oxalate ester label, a luciferin label, a luciferase label, and an aequorin label.

Illustrative examples of nuclear magnetic resonance contrasting agents include paramagnetic heavy metal nuclei such as Gd, Mn, and Fe.

The coupling of one or more molecules to antibodies is envisioned to include many chemical mechanisms, for instance covalent binding, affinity binding, intercalation, coordinate binding, and complexation

The covalent binding can be achieved either by direct condensation of existing side chains or by the incorporation of external bridging molecules. Many bivalent or polyvalent agents are useful in coupling protein molecules to other proteins, peptides or amine functions, etc. For example, the literature is replete with coupling agents such as carbodiimides, diisocyanates, glutaraldehyde, diazobenzenes, and hexamethylene diamines. This list is not intended to be exhaustive of the various coupling agents known in the art but, rather, is exemplary of the more common coupling agents.

In preferred embodiments, it is contemplated that one may wish to first derivatize the antibody, and then attach the toxin component to the derivatized product. Suitable cross-linking agents for use in this manner include, for example, SPDP (N-succinimidyl-3-(2-pyridyldithio)propionate), and SMPT, 4-succinimidyl-oxycarbonyl-α-methyl-α(2-pyridyldithio)toluene.

Radionuclides typically are coupled to an antibody by chelation. For example, in the case of metallic radionuclides, a bifunctional chelator is commonly used to link the isotope to the antibody or other protein of interest. Typically, the chelator is first attached to the antibody, and the chelator-antibody conjugate is contacted with the metallic radioisotope. A number of bifunctional chelators have been developed for this purpose, including the dithylenetriamine pentaacetic acid (DTPA) series of amino acids described in U.S. Pat. Nos. 5,124,471, 5,286,850 and 5,434,287, which are incorporated herein by reference. As another example, hydroxamic acid-based bifunctional chelating agents are described in U.S. Pat. No. 5,756,825, the contents of which are incorporated herein. Another example is the chelating agent termed p-SCN-Bz-HEHA (1,4,7,10,13,16-hexaazacyclo-octadecane-N,N′,N″,N′″,N″″,N′″″-hexaacetic acid) (Deal et al., J. Med. Chem. 42:2988, 1999), which is an effective chelator of radiometals such as ²²⁵Ac.

In yet other embodiments, the antibodies can be chimeric or humanized antibodies. As used herein, the term “chimeric antibody” refers to an antibody, that combines the murine variable or hypervariable regions with the human constant region or constant and variable framework regions. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications according to the invention.

In certain embodiments, the antibodies are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse have been grafted onto human framework sequences (referred to herein as “humanized antibodies”). Human antibodies directed against BACE or LRP can be generated using transgenic mice carrying parts of the human immune system rather than the mouse system.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Another type of the Group I or Group II agents of the present invention are peptide agents which are classified as antisense-peptide sequences. Antisense-peptide sequences are short peptides which are specifically designed to bind to a particular amino acid sequence. In general, such antisense peptide agents may be generated using methods known in the art, such as those described, for example, in Hurby et al., “Application of Synthetic Peptides: Antisense Peptides,” in Synthetic Peptides, A User's Guide, W. H. Freeman, N.Y., pp. 289-307 (1992) and Kaspczak et al., Biochemistry 28:9230-8 (1989).

An additional class of the agents of the invention are natural ligands of BACE or LRP that reduce or block the binding of the respective protein. As used herein, a natural ligand of BACE is defined as any substance which binds to BACE, such as soluble fragments of LRP containing the BACE binding site, e.g., the NPxY (SEQ ID NO:3) motifs. Likewise, a natural ligand of LRP is defined as any substance which binds to LRP, such as soluble fragments of BACE containing the LRP binding site, Such soluble fragments may be prepared by any suitable method known to those skilled in the art, such as the method of Davis et al., Nature 326:760-765 (1987), which involves deletion of the entire EGF domain. Moreover, soluble forms of the receptor may be formed by inserting a stop codon in front of the region of DNA encoding the cytoplasmic or transmembrane domain (Yokade et al., J. Cell. Biol. 117:39 (1992)).

The agents of the present invention may be used in vitro and/or in vivo to study interaction and binding LRP and BACE, and to reduce the rate of onset and/or ameliorate the duration and severity of Alzheimer's disease (i.e., to treat or prevent Alzheimer's disease). In addition, the agents of the present invention may be used in qualitative, quantitative and preparative assays and purification procedures to isolate, identify and facilitate the purification of BACE or LRP.

For in vivo use, the agents of the present invention may be provided to a patient as a means of reducing the amount or rate of BACE binding to LRP (Hayden, et al., Antiviral Res. 9:233-247 (1988)).

The present invention therefore provides pharmaceutical compositions comprising a Group I agent and/or a Group II agent. These pharmaceutical compositions may be administered orally, rectally, parenterally, intrathecally, intracistemally, intravaginally, intraperitoneally, topically (as by powders, ointments, drops or transdermal patch), bucally, or as an oral or nasal spray. As used herein, “pharmaceutically acceptable carrier” is intended to mean a non-toxic solid, semisolid or liquid filler, diluent, encapsulating material or formulation auxiliary of any type. The term “parenteral” as used herein refers to modes of administration which include intravenous, intramuscular, intrathecal, intraperitoneal, intrastemal, subcutaneous and intraarticular injection and infusion. One of ordinary skill will recognize that the choice of a particular mode of administration can be made empirically based upon considerations such as the particular disease state being treated; the type and degree of the response to be achieved; the specific agent or composition employed; the age, body weight, general health, sex and diet of the patient; the time of administration and rate of excretion of the agent or composition; the duration of the treatment; drugs (such as a secretase inhibitor or other Alzheimer's disease therapeutic) used in combination or coincidental with the specific composition; and like factors well known in the medical arts.

Pharmaceutical compositions of the present invention for parenteral injection may comprise pharmaceutically acceptable sterile aqueous or nonaqueous solutions, dispersions, suspensions or emulsions as well as sterile powders for reconstitution into sterile injectable solutions or dispersions just prior to use. Illustrative examples of suitable aqueous and nonaqueous carriers, diluents, solvents or vehicles include, but are not limited to, water, ethanol, polyols (such as glycerol, propylene glycol, polyethylene glycol, and the like), carboxymethylceuulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, parabens, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of the therapeutic agent or inhibitor, it is desirable to slow the absorption from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include, but are not limited to, capsules, tablets, pills, powders, and granules. In such solid dosage forms, the active compounds are preferably mixed with at least one pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents as appropriate.

Solid compositions of a similar type may also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Illustrative examples of embedding compositions which can be used include polymeric substances and waxes.

The active agents of Group I and/or Group II can also be in micro-encapsulated form, if appropriate, with one or more of the above-mentioned excipients.

Liquid dosage forms for oral administration include, but are not limited to, pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions may also contain adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

The agent or inhibitor can also be administered in the form of liposomes. As is known to those skilled in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the agent or inhibitor, stabilizers, preservatives, excipients, and the like. Preferred lipids are phospholipids and phosphatidyl cholines (lecithins), both natural and synthetic. (Methods to form liposomes are known in the art. See, e.g., Prescott, ed., METHODS IN CELL BIOLOGY, Volume XIV, Academic Press, New York, N.Y. (1976), p. 33 et seq.

The agents of the present invention can be formulated according to known methods to prepare pharmaceutically acceptable compositions, whereby these materials, or their functional derivatives, are combined in a mixture with a pharmaceutically acceptable carrier vehicle. Suitable vehicles and their formulation, inclusive of other human proteins, e.g., human serum albumin, are well known in the art. In order to form a pharmaceutically acceptable composition suitable for effective administration, such compositions will contain an effective amount of one or more agents of the present invention.

Additional pharmaceutical methods may be employed to control the duration of action. Controlled release preparations may be achieved through the use of polymers to complex or absorb the therapeutic agents of the invention. The controlled delivery may be exercised by selecting appropriate macromolecules (such as polyesters, polyamino acids, polyvinyl, pyrrolidone, ethylenevinylacetate, methylcellulose, carboxymethylcellulose, or protamine sulfate) and methods of incorporation in order to control release. Another possible method to control the duration of action by controlled release preparations is to incorporate antibodies into particles of a polymeric material such as polyesters, polyamino acids, hydrogels, poly(lactic acid) or ethylene vinyl acetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatine-microcapsules and poly(methylmethacylate) microcapsules, respectively, or in colloidal drug delivery systems, for example, liposomes, albumin microspheres, microemulsions, nanoparticles, and nanocapsules or in macroemulsions.

The pharmaceutical formulations of the present invention are prepared, for example, by admixing the active agent with solvents and/or carriers, optionally using emulsifiers and/or dispersants, while if water is used as the diluent, organic solvents may be used as solubilizing agents or auxiliary solvents. As described above, the excipients used include, for example, water, pharmaceutically acceptable organic solvents such as paraffins, vegetable oils, mono- or polyfunctional alcohols, carriers such as natural mineral powders, synthetic mineral powders, sugars, emulsifiers and lubricants.

One of ordinary skill will appreciate that effective amounts of the inventive therapeutic agents can be determined empirically and may be employed in pure form or, where such forms exist, in pharmaceutically acceptable salt, ester or prodrug form. The agonist or antagonist my be administered in compositions in combination with one or more pharmaceutically acceptable excipients. It will be understood that, when administered to a human patient, the total daily usage of the agents and compositions of the present invention will be decided by the attending physician within the scope of sound medical judgement. The specific therapeutically effective dose level for any particular patient will depend upon a variety of factors including the type and degree of the response to be achieved; the specific agent or composition employed; the age, body weight, general health, sex and diet of the patient; the time of administration, route of administration, and rate of excretion of the agent or composition; the duration of the treatment; drugs (such as a chemotherapeutic agent) used in combination or coincidental with the specific composition; and like factors well known in the medical arts.

Techniques of dosage determination are well known in the art for antibody and peptide agents. In general, it is desirable to provide a patient with a dosage of antibody or peptide agent in the range of from about 1 pg/kg to 10 mg/kg (body weight of patient). The therapeutically effective dose can be lowered if the agent of the present invention is additionally administered with another compound. As used herein, one compound is said to be additionally administered with a second compound when the administration of the two compounds is in such proximity of time that both compounds can be detected at the same time in the patient's serum.

For example, satisfactory results are obtained by oral administration of therapeutic dosages on the order of from 0.05 to 10 mg/kg/day, preferably 0.1 to 7.5 mg/kg/day, more preferably 0.1 to 2 mg/kg/day, administered once or, in divided doses, 2 to 4 times per day. On administration parenterally, for example by i.v. drip or infusion, dosages on the order of from 0.01 to 5 mg/kg/day, preferably 0.05 to 1.0 mg/kg/day and more preferably 0.1 to 1.0 mg/kg/day can be used. Suitable daily dosages for patients are thus on the order of from 2.5 to 500 mg p.o., preferably 5 to 250 mg p.o., more preferably 5 to 100 mg p.o., or on the order of from 0.5 to 250 mg i.v., preferably 2.5 to 125 mg i.v. and more preferably 2.5 to 50 mg i.v.

Dosaging may also be arranged in a patient specific manner to provide a predetermined concentration of an agent in the blood, as determined by the RIA technique. Thus patient dosaging may be adjusted to achieve regular on-going trough blood levels, as measured by RIA, on the order of from 50 to 1000 ng/ml, preferably 150 to 500 ng/ml.

The agents of the present invention are intended to be provided to a patient in an amount sufficient to reduce the amount or rate of binding of human BACE to LRP, i.e., and effective amount. An amount is said to be sufficient to “reduce the amount or rate of BACE binding” if the dosage, route of administration, etc. of the agent is sufficient to reduce the amount or rate of BACE binding to the LRP protein. Such an effect can be assayed, for example, by examining the onset of Alzheimer's disease symptoms occurring in vivo, by determining the level of Aβ or other BACE proteolytic product produced prior to and after the use of the agent(s), or by correlating in vitro blocking studies with predicted in vivo efficacy. In addition to APP, additional BACE substrates can be similarly assayed; for a review of BACE substrates see e.g., Gruninger-Leitch et al., J. Biol. Chem. 277(7):4687-4693, 2002.

The administration of the agents of the present invention may be for either prophylactic or therapeutic purpose. When provided prophylactically, the agent is provided in advance of any Alzheimer's disease symptoms. The prophylactic administration of the agent serves to prevent or reduce the rate of onset of symptoms. When provided therapeutically, the agent is provided at (or after) the onset of the appearance of symptoms of actual disease. The therapeutic administration of the agent serves to reduce the severity and duration of Alzheimer's disease.

The present invention further includes the use of the agents of the present invention in diagnostic applications. The Group I agents of the present invention can be used to detect the presence of LRP in a test sample. The Group II agents of the present invention can be used to detect the presence of BACE in a test sample.

Conditions for incubating an agent with a test sample vary. Incubation conditions will depend on factors such as the type of agent, format, and detection system employed for the assay, as well as the nature of the test sample used in the assay. For example, condition will vary slightly when a whole antibody, a single chain antibody, a F(ab) fragment, or a peptide agent is used. One skilled in the art will recognize that any one of the commonly available immunological assay formats (such as radioimmunoassays, enzyme-linked immunosorbent assays, diffusion based ouchterlony, or rocket immunofluorescent assays) can readily be adapted to employ the antibodies of the present invention. Examples of such assays can be found in T. Chard, An Introduction to Radioimmunoassay and Related Techniques, Elsevier Science Publishers, Amsterdam, The Netherlands (1986); G. R. Bullock et al., Techniques in Immunocytochemistry, Academic Press, Orlando, Fla. Vol. 1 (1982), Vol. 2 (1983), Vol. 3 (1985); and P. Tijssen, P., Practice and Theory of Enzyme Immunoassays: Laboratory Techniques in Biochemistry and Molecular Biology, Elsevier Science Publishers, Amsterdam, The Netherlands (1985).

In one embodiment of the above-described method, the agent of the present invention is immobilized on a solid support for use in the diagnostic assay. Illustrative examples of such solid supports include, but are not limited to, plastics such as polycarbonate, complex carbohydrates such as agarose and sepharose, and acrylic resins, such as polyacrylamide and latex beads. Techniques for coupling agents such as antibodies, peptides and the like to such solid supports are well known in the art, as described, for example, in D. M. Weir et al., Handbook of Experimental Immunology, 4th Ed., Blackwell Scientific Publications, Oxford, England, Chapter 10 (1986) and W. D. Jacoby et al., Meth. Enzym. 34, Academic Press, N.Y. (1974).

Additionally, one or more of the agents of the present invention which is used in one of the above-described methods can be detectably labeled prior to use, for example, through the use of radioisotopes, affinity labels (such as biotin, avidin, etc.), enzymatic labels (such as horse radish peroxidase, alkaline phosphatase, etc.) fluorescent labels (such as FITC, Cy3 or rhodamine, etc.), paramagnetic atoms, etc. Procedures for accomplishing such labeling are well-known in the art (L. A. Stemberger et al., J. Histochem. Cytochem. 18:315 (1970); E. A. Bayer et al., Meth. Enzym. 62:308 (1979); E. Engval et al., Immunol. 109:129 (1972); and J. W. Goding, J. Immunol. Meth. 13:215 (1976)).

The materials used in the inventive assays are ideally suited for the preparation of a kit. For example, the present invention provides a compartmentalized kit to receive in close confinement, one or more containers which comprises: a) a first container comprising an agent capable of binding to the LRP binding site or BACE; and b) one or more other containers comprising one or more of the following: wash reagents and reagents capable of detecting the presence of bound agents from the first container.

As used herein, a compartmentalized kit includes any kit in which reagents are contained in separate containers. Illustrative examples of such containers include, but are not limited to, small glass containers, plastic containers or strips of plastic or paper. Particularly preferred types of containers allow the skilled worker to efficiently transfer reagents from one compartment to another compartment such that the samples and reagents are not cross-contaminated and the agents or solutions of each container can be added in a quantitative fashion from one compartment to another. Such containers include, but are not be limited to, a container which will accept the test sample, a container which contains one or more of the agents of the present invention used in the assay, containers which contain wash reagents (such as phosphate buffered saline, Tris-buffers, etc.), and containers which contain the reagents used to detect the bound agent.

The types of detection reagents which can be used in the above described kits include, but are not limited to, labeled secondary agents, or in the alternative, if the primary agent is labeled, enzymatic or agent binding reagents which are capable of reacting with the labeled agent. One skilled in the art will readily recognize that the agents of the present invention can be readily incorporated into one of the established kit formats which are well known in the art.

A further aspect of the present invention concerns DNA molecules which encode for the polypeptide and antibody agents of Group I and/or Group II. The starting nucleotide molecules can be obtained by the person skilled in the art using known methods. Moreover, the DNA molecules, where the amino acid sequence is known, may be produced synthetically or by amplification methods such as PCR (Sambrook et al., Molecular Cloning: A Laboratory Manual, Cold Spring Harbor Laboratory Press (1989)). The DNA sequences of the present invention include not only the actual nucleotide sequence used by the organism from which the receptor protein is derived but also includes all degenerate forms which encode a peptide with the desired sequence. The invention includes DNA sequences which have been modified utilizing methods known in the art, such as those generated by mutation, deletion, transposition or addition.

The present invention further includes DNA vectors which contain the DNA sequences described above and below. In particular, these may be vectors in which the DNA molecules described are functionally linked to control sequences which allows expression of the corresponding polypeptides. These are preferably plasmids which can be replicated and/or expressed in prokaryotes such as E. coli and/or in eukaryotic systems such as yeasts or mammalian cell lines. These vectors may also be mammalian viral vectors which can be replicated and/or expressed in eukaryotes such as mammalian cell lines and in the human patient, as “host,” for integration into the cellular genome of the patient and expression as genetic therapy systems.

The invention also includes host organisms transformed with the above vectors. Expression in prokaryotes and eukaryotes may be carried out using techniques known in the art. The DNA sequences according to the invention may be expressed as fusion polypeptides or as intact, native polypeptides. Fusion proteins may advantageously be produced in large quantities. They are generally more stable than the native polypeptide and are easy to purify. The expression of these fusion proteins can be controlled by normal host DNA sequences.

The prerequisite for producing intact native polypeptides using E. coli is the use of a strong, regulatable promoter and an effective ribosome binding site. Promoters which may be used for this purpose include the temperature sensitive bacteriophage λp_{L}-promoter, the tac-promoter inducible with IPTG or the T7-promoter. Numerous plasmids with suitable promoter structures and efficient ribosome binding sites have been described, such as for example pKC30 (λp_{L}; Shimatake and Rosenberg, Nature 292:128 (1981), pKK173-3 (tac, Amann and Brosius, Gene 40:183 (1985)) or pET-3 (T7-promoter (Studier and Moffat, J. Mol. Biol. 189:113 (1986)).

A number of other suitable vector systems for expressing the DNA according to the invention in E. coli are known from the prior art and are described, for example, in Sambrook et al., Molecular Cloning: A Laboratory Manual, Cold Spring Harbor Laboratory Press (1989)).

Suitable E. coli strains which are specifically tailored to a particular expression vector are known to those skilled in the art (Sambrook et al., Molecular Cloning: A Laboratory Manual, Cold Spring Harbor Laboratory Press (1989)). The experimental performance of the cloning experiments, the expression of the polypeptides in E. coli and the working up and purification of the polypeptides are known and are described, for example, in Sambrook et al., Molecular Cloning: A Laboratory Manual, Cold Spring Harbor Laboratory Press (1989). In addition to prokaryotes, eukaryotic microorganisms such as yeast may also be used.

For expression in yeast, the plasmid YRp7 (Stinchcomb et al. Nature 282:39 (1979); Kingsman et al., Gene 7:141 (1979); Tschumper et al., Gene 10:157 (1980)) and the plasmid YEp13 (Bwach et al., Gene 8:121-133 (1979)) are used, for example. The plasmid YRp7 contains the TRP 1-gene which provides a selection marker for a yeast mutant (e.g., ATCC No. 44076) which is incapable of growing in tryptophan-free medium. The presence of the TRP 1 defect as a characteristic of the yeast strain used then constitutes an effective aid to detecting transformation when cultivation is carried out without tryptophan. The same is true with the plasmid YEp13, which contains the yeast gene LEU-2, which can be used to complete a LEU-2-minus mutant.

Other suitable marker genes for yeast include, for example, the URA3- and HIS3-gene. Preferably, yeast hybrid vectors also contain a replication start and a marker gene for a bacterial host, particularly E. coli, so that the construction and cloning of the hybrid vectors and their precursors can be carried out in a bacterial host. Other expression control sequences suitable for expression in yeast include, for example, those of PHO3— or PHO5-gene.

Other suitable promoter sequences for yeast vectors contain the 5′-flanking region of the genes of ADH I (Ammerer, Methods of Enzymology 101: 192-210 (1983)), 3-phosphoglycerate kinase (Hitzeman et al., J. Biol. Chem. 255:2073 (1980)) or other glycolytic enzymes (Kawaski and Fraenkel, BBRC 108:1107-1112 (1982)) such as enolase, glycerinaldehyde-3-phosphate-dehydrogenase, hexokinase, pyruvate-decarboxylase, phosphofructokinase, glucose-6-phosphate isomerase, phosphoglucose-isomerase and glucokinase. When constructing suitable expression plasmids, the termination sequences associated with these genes may also be inserted in the expression vector at the 3′-end of the sequence to be expressed, in order to enable polyadenylation and termination of the mRNA.

Generally, any vector which contains a yeast-compatible promoter and origin replication and termination sequences is suitable. Thus, hybrid vectors which contain sequences homologous to the yeast 2μ plasmid DNA may also be used. Such hybrid vectors are incorporated by recombination within the cells of existing 2μ-plasmids or replicate autonomously.

In addition to yeasts, other eukaryotic systems may, of course, be used to express the polypeptides according to the invention. Since post-translational modifications such as disulphide bridge formation, glycosylation, phosphorylation and/or oligomerization are frequently necessary for the expression of biologically active eukaryotic proteins by means of recombinant DNA, it may be desirable to express the DNA according to the invention not only in mammalian cell lines but also insect cell lines.

Functional prerequisites of the corresponding vector systems comprise, in particular, suitable promoter, termination and polyadenylation signals as well as elements which make it possible to carry out replication and selection in mammalian cell lines. For expression of the DNA molecules according to the invention it is particularly desirable to use vectors which are replicable both in mammalian cells and also in prokaryotes such as E. coli.

Vectors derived from viral systems such as SV40, Epstein-Barr-virus, etc., include, for example, pTK2, pSV2-dhfv, pRSV-neo, pKO-neo, pHyg, p205, pHEBo, etc. (Sambrook et al., Molecular Cloning: A Laboratory Manual, Cold Spring Harbor Press, N.Y. (1989)).

After transformation in suitable host cells, e.g. CHO cells, corresponding transformed cells may be obtained with the aid of selectable markers (thymidine-kinase, dihydrofolate-reductase, green fluorescent protein, etc.) and the corresponding polypeptides are isolated after expression. The host cells suitable for the vectors are known, as are the techniques for transformation (micro-injection, electroporation, calcium phosphate method, etc.) as described, for example, in Sambrook et al., Molecular Cloning: A Laboratory Manual, Cold Spring Harbor Press, N.Y. (1989).

For cloning corresponding DNA fragments in prokaryotic or eukaryotic systems, the selected vector may be cut, for example, with a restriction endonuclease and, optionally after modification of the linearized vector thus formed, an expression control sequence equipped with corresponding restriction ends is inserted. At the 3′-end (in the direction of translation) the expression control sequence contains the recognition sequence of a restriction endonuclease, so that the vector already containing the expression control sequence is digested with the said restriction enzyme and the DNA molecule according to the invention, provided with ends which fit, can be inserted. It is advantageous to cleave the vector which already contains the expression control sequence with a second restriction endonuclease inside the vector DNA and to insert the DNA molecule provided with the correct ends into the vector fragment produced. The techniques required are described, for example, by Sambrook et al. Molecular Cloning: A Laboratory Manual Cold Spring Harbor Press. N.Y. (1989).

Apart from the DNA molecules specified, the invention also relates to processes for preparing the vectors described herein, particularly expression vectors. These vectors are characterized in that a DNA provided with corresponding ends and coding for a functional derivative or a fragment of the LRP protein or the BACE protein is inserted into a vector DNA cut with restriction endonucleases and containing the expression control sequences described by way of example, in such a way that the expression control sequences regulate the expression of the DNA inserted. The peptides and antibody agents of the present invention which are obtained by the expression of recombinant DNA or from the native receptor molecule may, of course, also be derivatized by chemical or enzymatic processes.

The invention also relates in some aspects to the identification and testing of candidate agents and molecules that can reduce the binding of BACE to LRP. The binding-reduction polypeptides and fragments of the invention can be screened for reducing binding using the same type of assays as described herein (e.g., the FRET assays described in the Examples section). Using such assays, the binding-reduction polypeptides and/or nucleic acid molecules that have the best inhibitory activity can be identified. It is understood that any mechanism of action described herein for the binding-reduction polypeptides and/or nucleic acids is not intended to be limiting, and the scope of the invention is not bound by any such mechanistic descriptions provided herein.

The binding-reduction molecules or agents of the invention also include small molecules and/or chemicals that reduce BACE binding to LRP. The binding reduction molecules may be identified using the assays provided herein, including those in the Examples section. For example, a candidate agent or compound may be tested for its ability to reduce BACE binding to LRP (e.g., an agent which selectively inhibits the level or effect of binding of BACE to LRP). To test the ability of an agent or compound to reduce this specific binding, BACE and LRP may be contacted with a candidate binding-reduction compound or agent and the level of binding of BACE to LRP can be compared to the level of binding of BACE to LRP in the absence of the candidate binding-reduction compound or agent.

The invention further provides efficient methods of identifying pharmacological agents or lead compounds for agents and molecules that reduce BACE binding to LRP. Generally, the screening methods involve assaying for compounds which modulate (up- or down-regulate) the level of binding between BACE and LRP, respectively. As will be understood by one of ordinary skill in the art, the screening methods may measure the level of binding between the molecules directly, such as by using the methods employed in the Examples. In addition, screening methods may be utilized that measure a secondary effect of the binding of BACE to LRP, for example the level of production of an APP cleavage product (e.g., Aβ) in a cell or tissue sample.

A wide variety of assays for pharmacological agents can be used in accordance with this aspect of the invention, including, labeled in vitro protein-protein binding assays, electrophoretic mobility shift assays, immunoassays, cell-based assays such as two- or three-hybrid screens, expression assays, etc. The assay mixture comprises a candidate pharmacological agent. Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection.

Candidate agents useful in accordance with the invention encompass numerous chemical classes, although typically they are organic compounds. Preferably, the candidate pharmacological agents are small organic compounds, i.e., those having a molecular weight of more than 50 yet less than about 2500, preferably less than about 1000 and, more preferably, less than about 500. Candidate agents comprise functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

It is contemplated that cell-based assays as described herein can be performed using cell samples and/or cultured cells. Biopsy cells and tissues as well as cell lines grown in culture are useful in the methods of the invention.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

An exemplary binding assay is described herein, which may be used to identify candidate agents that modulate the binding of BACE to LRP. In general, the mixture of the foregoing assay materials is incubated under conditions whereby, but for the presence of the candidate pharmacological agent, BACE binds to LRP, although in some embodiments the candidate agent may be one that increases the binding between BACE and LRP. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the presence or absence of specific binding between BACE and LRP is detected by any convenient method available to the user. For cell-free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximum signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromatographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

Detection may be effected in any convenient way for cell-based assays such as two- or three-hybrid screens. For cell-free binding assays, at least one of the components usually comprises, or is coupled to, a detectable label. A wide variety of labels can be used, such as those that provide direct detection (e.g., radioactivity, luminescence, optical or electron density, energy transfer, etc.) or indirect detection (e.g., epitope tag such as the FLAG or myc epitopes, enzyme tag such as horseradish peroxidase, etc.). The label may be bound to BACE and/or LRP (covalently or non-covalently) or may be incorporated into or bound to the candidate agent.

A variety of methods may be used to detect the label, depending on the nature of the label and other assay components. For example, the label may be detected while bound to the solid substrate or subsequent to any separation from the solid substrate. Labels may be directly detected through optical or electron density, radioactive emissions, nonradiative energy transfers, etc. or indirectly detected with antibody conjugates, strepavidin-biotin conjugates, etc. A variety of methods for detecting the labels are well known in the art.

The present invention is described in further detail in the following non-limiting examples.

### EXAMPLES

BACE (β-site of APP-cleaving enzyme) is a type I membrane-associated aspartyl protease that cleaves APP (Hussain et al., 1999; Sinha et al., 1999; Vassar et al., 1999; Yan et al., 1999). Besides APP, the few BACE substrates that have been identified include the APP homologues APLP1 and 2 (Li and Sudhof, 2004) and a membrane-bound sialyltransferase (Kitazume et al., 2001). Post-translational processing of BACE involves N-glycosylation, removal of its prodomain by a furin-like protease, and further complex glycosylation (Bennett et al., 2000; Capell et al., 2000; Creemers et al., 2001). After glycosylation, BACE co-traffics with APP and is rapidly transported to the Golgi apparatus and distal secretory pathway (Creemers et al., 2001). Measurable amounts of APP and BACE are present on the plasma membrane (Huse et al., 2000; Riddell et al., 2001; Kinoshita et al., 2003b) and in lipid rafts (Riddell et al., 2001; Cordy et al., 2003; Ehehalt et al., 2003). BACE and APP are internalized from the cell surface to early endosomes and cycle between the cell membrane and endosomes (Huse et al., 2000; Walter et al., 2001; Kinoshita et al., 2003b).

The low density lipoprotein receptor-related protein, LRP, is a type I integral membrane protein with a 515 kDa extracellular a-chain non-covalently bound to the 85 kDa membrane-spanning b-chain. Multiple intracellular adaptor and scaffolding proteins bind LRP's 100 amino acid cytoplasmic tail (Herz and Strickland, 2001; Li et al., 2001).; its four extracellular binding domains mediate endocytosis of a wide array of ligands, including several of potential importance for pathophysiology: APP, apolipoprotein E and α2-macroglobulin (Kounnas et al., 1995; Herz and Strickland, 2001; Li et al., 2001). In particular, LRP's ligand binding domains interact with KPI-containing forms of APP; a heterotrimeric complex of APP, LRP, and the adaptor protein Fe65 mediates APP internalization (Kounnas et al., 1995; Trommsdorff et al., 1998; Ulery et al., 2000; Kinoshita et al., 2001; Rebeck et al., 2001; Pietrzik et al., 2002). In addition to its role in endocytosis, LRP has an interesting pattern of proteolysis that parallels APP in some ways. Ectodomain shedding of LRP has been described (Quinn et al., 1999) and proteolysis of LRP by matrix metalloproteases was recently reported (Rozanov et al., 2004); MT1-MMP also cleaves APP (Higashi and Miyazaki, 2003) and the postulated alpha-secretases of the ADAM-family are also metalloproteinases. Furthermore, and as with APP, γ-secretase cleavage of LRP leads to release of the LRP intracellular domain (LRP-ICD), which can translocate to the nucleus and interact with Tip60 (May et al., 2002; Kinoshita et al., 2003a).

Given these similarities, we examined whether LRP and BACE interact. Using both a FRET-based assay of protein proximity and co-immunoprecipitation (co-IP) we demonstrate that the LRP-ICD interacts with BACE and that this interaction seems to take place in lipid rafts on the cell surface. Surprisingly, BACE-LRP interaction does not appear to enhance BACE endocytosis from the cell surface but instead leads to LRP cleavage and release of the LRP intracellular domain from the membrane. Taken together, these data suggest that a close interaction between BACE and LRP on the cell surface makes LRP into a BACE substrate.

### EXAMPLE 1

Materials and Methods

Generation of Expression Constructs of LRP and BACE

The generation of the LC-myc construct that encodes the light chain (LC) of LRP, tagged with myc at its C-terminus, has been described previously (Mikhailenko et al., 2001). The NPxY mutant of the LC-myc was generated by substitutions of asparagine and tyrosine to alanines in the first, the second, or the both of the two NPxY (SEQ ID NO:3) motifs in the cytoplasmic domain of the LC-myc using the Transformer site-directed mutagenesis kit (Clontech, Palo Alto, Calif.) and confirmed by sequencing. To make the LRP165-myc construct, LC-myc was digested with PstI and the band containing the vector and the carboxyl terminal of LRP (encoding 165 amino acids) was extracted and self-ligated.

The details of the BACE constructs have been described elsewhere (Kinoshita et al., 2003b). In brief, BACE was cloned by PCR from a whole human brain cDNA library (Quick Clone cDNA; Clontech, Palo Alto, Calif.) using these primers: 5′-AGCCACCAGCACCACCAGACTTG-3′ (SEQ ID NO:1), 5′-ACTGGTTGGTAACCTCACCCATTA-3′ (SEQ ID NO:2). The PCRed BACE clone was inserted into pcDNA3.1/V5/His-Topo vector (Invitrogen, Carlsbad, Calif.) (BACE-V5). BACE was cut out from the BACE-V5 at HindIII and SacII sites and inserted into the pEGFP-N1 vector (Clontech) to make BACE-GFP construct. To make the N-terminally myc tagged BACE construct, the PCRed BACE fragment containing XhoI and XbaI sites at the ends, was inserted into pSecTagN2myc plasmid (a gift from Dr. I. Mikhailenko, American Red Cross, Rockville, Md.). Authenticity of the PCR-generated constructs was confirmed by DNA sequencing. The Fe65-myc clone has been described previously (Kinoshita et al., 2001) and pcDNA3.1-mDab1 is a generous gift from Dr. J. Hertz (University of Texas Southwestern Medical Center, Dallas, Tex.).

Cell Culture Conditions and Transient Transfection

H4 cells derived from human neuroglioma cells are used in this study. H4 cells were cultured in OPTI-MEMI with 10% fetal bovine serum. Transient transfection of the cells was performed using a liposome-mediated method (FuGene 6; Roche Molecular Biochemicals, Indianapolis, Ind.). For immunocyotochemistry, cells were split into 4-well chambers 1 day before the transfection. First a mixture of 1 μg of plasmid DNA and 3 μl of FuGene6 was made in 100 μl of Dulbecco's modified Eagle's medium and left for 15-30 min at room temperature. Then 25 μl of it was added to the medium in each well. The incubation time was from 24 hours to 48 hours. Double transfection of LRP and BACE constructs was performed in the same way. For the co-immunoprecipitation experiments, H4 cells were split into 100 mm² dishes, then co-transfected with BACE-V5 and LC-myc (wild type or NPxY mutant type) constructs. They were harvested 24 hours after transfection.

Immunohistochemistry

Immunostaining was done on the cells 24-48 h post-transfection. Cells were fixed in 4% paraformaldehyde for 10 min, washed in Tris-buffered saline (TBS pH7.3) and permeabilized by 0.5% TritonX-100 for 20 min, and blocked with 1.5% normal goat serum for 1 h. To detect the localization of LC and BACE, cells transfected with LC-myc and BACE-V5 were immunostained by rabbit anti-myc antibody (Upstate 1:1000) and mouse anti-V5 antibody (Invitrogen, Carlsbad, Calif.: 1:5000). Cells were then washed three times in TBS and labeled by FITC-conjugated anti-rabbit antibody and Cy3-conjugated anti-mouse antibody (Jackson Immunoresearch, West Grove, Pa., 10 μg/ml) for 1 h at room temperature. An alternative method was also performed to confirm the result. H4 cells co-transfected with LC-myc and BACE-GFP were immunostained with mouse anti-myc antibody (Invitrogen, 1:1000), then labeled by Cy3. Immunostained cells were coverslipped and mounted for confocal or two photon microscopic imaging.

To detect the co-localization of BACE, LC with Golgi organelle marker GM130 (BD Transduction Laboratories, San Diego, Calif.), H4 cells were co-transfected with BACE-GFP and LC-myc, then immunostained with rabbit anti-myc antibody (Upstate Biotechnology, Lake Placid, N.Y., visualized by Cy3) and mouse anti-GM130 antibody (1:200, visualized by Cy5). To detect the co-localization of BACE, LC with endosomal marker EEA, H4 cells were co-transfected with BACE-V5 and LC-myc, and then immunostained with anti-V5 (mouse, visualized by Cy5) and anti-myc (rabbit, visualized by Cy3), then washed thoroughly, and FITC-conjugated anti-EEA antibody (BD Transduction Laboratories) was applied. The immunostained cells were observed with the appropriate filters using a confocal microscopy.

Co-Immunoprecipitation

Immunoprecipitation experiments were carried out with BioMag Goat anti-Mouse IgG (PerSeptive Biosystems, Framingham, Mass.). For the pulldown of BACE-V5, the magnetic beads were pre-incubated with anti-V5 antibody (10 μg/ml) overnight at 4° C. on a rocking platform, then BACE-V5 and (wild or mutant) LC-myc co-expressing H4 cell lysates were precipitated with BioMag for 2 h at 22° C. The beads were washed four times in lysis buffer. Two other conditions served as negative controls. For one negative control, we incubated the beads in TBS without adding the anti-V5 antibody, then, cell lysates were applied to the beads. For another negative control, we incubated the beads in the presence of anti-V5 antibody, but the lysates were not applied. Next, the sample buffer was added to the magnet beads of each condition, and then denatured at 100° C. for 3 min, then centrifuged. The supernatant was collected, and proteins were separated on 10-20% Tris-glycine polyacrylamide gels (Novex, San Diego, Calif.) under denaturing, reducing conditions and transferred to immobilon-P membrane (Millipore, Bedford, Mass.). Membranes were blocked in 5% non-fat dried milk. For detection of LC-myc, rabbit anti-myc antibody (1 μg/ml) was used. Secondary antibody conjugated to horse radish peroxidase was applied and visualized by chemiluminescence. To confirm this result, we performed a pulldown of LC-myc using mouse monoclonal anti-myc antibody (10 μg/ml). A rabbit polyclonal anti-BACE antibody against the N-terminus of BACE (Affinity Bioreagents, Golden, Colo.) was used to probe for BACE in the LC immunoprecipitates.

FRET Measurements using Fluorescence Lifetime Imaging Microscopy (FLIM)

FRET is observed when two fluorophores are in very close proximity, i.e., <10 nm. FRET measurements using FLIM relied on the observation that fluorescence lifetime (the time of fluorophore emission after brief excitation, measured in picoseconds) are shorter in the presence of a FRET acceptor. We recently developed a new FLIM technique that can quantitate protein-protein interactions using multiphoton microscopy (Bacskai et al., 2003; Berezovska et al., 2003). A mode-locked Ti-sapphire laser (Spectra Physics) sends a femtosecond pulse every 12 nsec to excite the fluorophore. Images were acquired using a BioRad Radiance 2000 multiphoton microscope. We used a high-speed Hamamatsu MCP detector and hardware/software from Becker and Hickl (Berlin, Germany) to measure fluorescence lifetimes on a pixel-by-pixel basis. Donor fluorophore (GFP or FITC) lifetimes were fit to two exponential decay curves to calculate the fraction of fluorophores within each pixel that either interact, or do not interact, with an acceptor. As a negative control, GFP or FITC lifetime was measured in the absence of acceptor (Cy3).

Results

Localization of LC and BACE

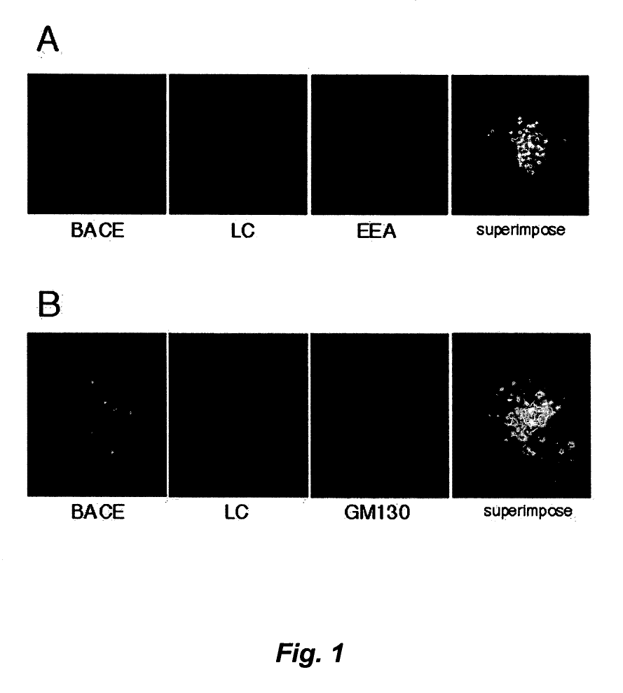

We first tested the localization of BACE and LC in co-expressing H4 cells. Double immunostaining was performed. When expressed individually, both LC and BACE were localized mainly in punctate structures in the cells; LC-positive structures largely overlapped with BACE-positive structures when they were co-expressed. To see what subcellular structures they are mostly co-localized in, we immunostained the co-expressing H4 cells with the organelle markers GM130 and EEA. As shown in FIG. 1A, LC and EEA showed substantial overlap, suggesting that LC is mainly localized in the endosomal compartment. BACE is colocalized with LC in the endosomal compartment, and is also present in the secretory pathway. However, LC is not co-localized with the Golgi marker, GM130. The result of the immunohistochemistry suggests that LC and BACE are mainly co-localized in the endosomal compartment, rather than in the secretory pathways.

Co-Immunoprecipitation of BACE and LC

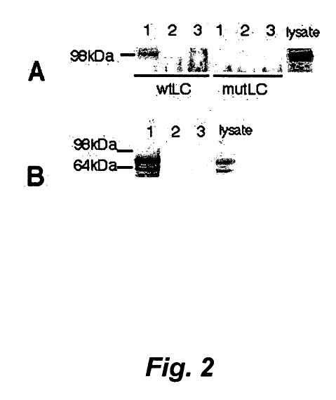

From the immunohistochemical experiments that showed robust co-localization, we hypothesized that there may be a direct interaction between LC and BACE in the endosomal compartments. To test whether LC interacts with BACE, we immunoprecipitated LC and BACE co-expressing H4 cell extracts with anti-V5 antibody to pull down BACE, and probed with an anti-myc antibody probing for LC. An immunoreactive band of approximately 100 kDa (Mikhailenko et al., 2001) was detected only in the sample lane (lane 1) and the lysates lane (FIG. 2A). Control precipitates from samples lacking anti-V5 antibody (lane 2) or cell extract (lane 3) did not contain this band. Furthermore, purified mutant form of LC containing both mutant NPxY motifs was not immunoprecipitated by the anti-V5 antibody (FIG. 2A), suggesting that double mutation of the two NPxY (SEQ ID NO:3) motifs interfered with the interaction between BACE and LC.

In order to confirm this interaction, the complementary pull down experiment was performed. LC immunoprecipitates were probed by an anti-BACE antibody. As seen in FIG. 2B, the doublet bands of approximately 60 kDa and 80 kDa were detected only in lane 1, and in the lysate lane, suggesting that BACE is present in the LC immunoprecipitates. The size of these bands are consistent with BACE containing V5 and His tags.

To further assess whether the BACE-LRP interaction was physiologically relevant, we immunoprecipitated BACE from human brain homogenate, and were able to detect LRP (light chain) immunoreactivity.

Interaction of LC and BACE by FLIM analysis

We next used an alternative technique to probe protein-protein proximity to test the idea that the LRP-BACE interaction detected by co-immunoprecipitation occurs in specific cell compartments, and to further evaluate the biochemical parameters of this interaction. We utilized a morphology based new FRET technique that can reveal protein-protein interactions in intact cells, fluorescence lifetime imaging microscopy (FLIM). Fluorescence lifetime is influenced by the surrounding microenvironment and is shortened in the immediate vicinity of a FRET acceptor molecule. The degree of lifetime shortening can be displayed with very high spatial resolution in a pseudo-color-coded image.

As shown in FIG. 1, double immunostaining showed subcellular compartment colocalization of BACE and LRP in endosomal compartments, but this does not necessarily imply a close interaction. Therefore, in order to further confirm the interaction and determine the interaction site, we used FLIM to analyze the lifetime of the donor fluorophore in intact cells. We measured changes in the lifetime of the donor fluorophore (either FITC or GFP) under different experimental conditions (Table 1).

TABLE 1
FLIM assay demonstrates close proximity between LC and BACE

| | | Lifetime | | Significance |
|---|---|---|---|---|
| | | (picoseconds) | | (compared to |
| Donor | Acceptor | (Mean ± SD) | n | the control) |
| LC-GFP | none | 2164 ± 10 | 5 | — |
| LC-GFP | BACE-V5 | 1165 ± 8 | 5 | p < 0.0001 |
| | (Cy3) | | | |
| LC-GFP | Myc-BACE | 2247 ± 102 | 5 | NS |
| | (Cy3) | | | |
| BACE-GFP | None | 2284 ± 20 | 6 | — |
| BACE-GFP | LRP165myc | 2183 ± 69 | 6 | p = 0.012 |
| | (Cy3) | | | |
| BACE-GFP | LC-myc (Cy3) | 1756 ± 50 | 4 | p = 0.0004 |

Table 1 shows the summary data of the FLIM assay for LC and BACE proximity. If there is no interaction, specific lifetimes of the fluorophore are observed as seen in the negative controls in the absence of the acceptor fluorophore. Statistically shorter lifetimes between LC and BACE regardless of the combination of fluorophore at their C-terminus suggest that there is FRET between them, however, if the fluorophore is located at the N-terminus of BACE (myc-BACE), no lifetime change is observed. The truncated form of LRP, LRP165, which lacks almost the entire extracellular domain of LC, showed a statistically significantly decreased degree of interaction.

Our negative control (in the absence of an acceptor fluorophore) showed that the lifetime of FITC (conjugated to IgG, hereafter referred to simply as FITC) alone is 2300-2400 psec, and GFP alone 2100-2200 psec. If there is no interaction, specific lifetimes of the fluorophore are observed as seen in the negative controls in the absence of acceptor fluorophore. Statistically shorter lifetimes (2164+/−10 in the LC-GFP alone, 1165+/−8 in the sample, P<0.0001) between LC-GFP and BACE-V5 (labeled by Cy3, at its C-terminus) suggest that there is FRET between them. In order to test the idea that the decrease in lifetime observed in the LC-GFP/BACE-V5 FLIM assay was due to FRET, we performed an additional control. LC-GFP and Myc-BACE co-transfected cells were immunostained with anti-myc antibody, then labeled with Cy3. In this experiment, the myc tag was at the N-terminus of BACE, across the membrane (and, therefore, too distant from the C-terminal GFP on LC-GFP to be detected by FRET). Although there was striking colocalization at the light level, no lifetime change was observed (2247+/−102 in the LC-GFP in the presence of Myc-BACE). This experiment demonstrates the specificity of the proximity assay in this FLIM-based method of measuring FRET.

Next, we tested the various mutants of LC to see their effect on the BACE-LRP interaction, in order to identify possible interaction sites between them. First, the truncated form of LC, LRP 165, was used to examine the effect of the extracellular domain on the interaction with BACE. LRP165, which lacks almost the entire extracellular domain of LC, showed a statistically significant decrease in the FLIM assay, compared to LC, suggesting that the extracellular domain contributes to the tight interaction, perhaps by stabilizing the conformation.

Second, we tested the effect of NPxY mutants on the interaction with BACE. When both of the NPxY (SEQ ID NO:3) motifs of the LC construct are mutated, the interaction between BACE and LC decreased substantially, suggesting little of no interaction, consistent with the result from the co-immunoprecipitation experiment (Table 2).

We then examined each NPxY mutation individually. When the 2^{nd} NPxY domain was mutated, LC interacted with BACE to the same degree as the wild type LC, suggesting that it is not a major determinant of LRP-BACE interactions. Mutation of the 1^{st} NPxY domain diminished the interaction between LRP and BACE (2282+/−51 psec for NPxY mutant vs 2181+/−61 psec for wild type, p<0.02) but did not completely ablate the interaction. Thus interactions depend on both the extracellular domain of LC (as noted by comparison of LC with LRP165) and the intracellular NPxY (SEQ ID NO:3) domain, especially the 1^{st} NPxY (SEQ ID NO:3) (Table 2).

TABLE 2
Analysis of the contribution of LRP NPxY domains to LRP-BACE interactions

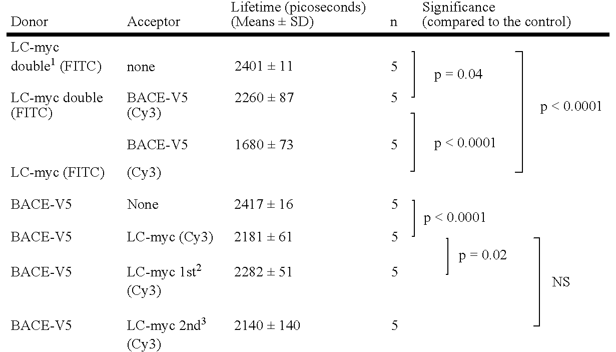

¹In this construct, both NPxY (SEQ ID NO:3) domains are mutated to APxA (SEQ ID NO:4).
²In this construct, the first NPxY (SEQ ID NO:3) domain is mutated to APxA (SEQ ID NO:4).
³In this construct, the second NPxY (SEQ ID NO:3) domain is mutated to APxA (SEQ ID NO:4).

There are several scaffold/adaptor proteins known for the interaction of LRP; Fe65 (Trommsdorffet al., 1998), mammalian disabled 1 (mDabl)(Trommsdorffet al., 1998), etc. If the NPxY (SEQ ID NO:3) motifs are responsible for the interaction between LRP and BACE, these adaptor proteins may play a role in the interaction. For example, we have earlier demonstrated that LRP and Fe65 interact using a FRET based cell assay (Kinoshita 2001). We therefore examined the possibility that these molecules may interact with BACE by the FRET assay. However, we did not detect any FRET between BACE and Fe65, or between BACE and mDabl, suggesting that there may be other players in the interaction via NPxY.

Discussion

In the present study, we demonstrate that BACE interacts with the light chain of LRP, a multi-functional endocytic receptor. The hypothesis that LRP and BACE interact is supported by co-localization, co-immunoprecipitation, FRET-based proximity assays and the demonstration of specific sites that are critical for the interactions. The possible interaction sites on LRP include the cytoplasmic NPxY (SEQ ID NO:3) motifs (especially the 1^{st} motif), and the extracellular domain of the light chain may also enhance LRP-BACE interactions, since LRP 165 did not show a tight interaction with BACE.

BACE interacts with an increasing number of other proteins. Sialyltransferase is reported to be another substrate of BACE (Kitazume et al., 2001; Kitazume et al., 2003). Nicastrin (Hattori et al., 2002) is found to activate β-secretase activity of BACE in the APP, BACE, PS1 and nicastrin complex. A family of GGA adapter proteins has been shown to directly interact with the BACE tail in a dileucine-dependent manner (He et al., 2002). Recently, the phospholipid scramblase 1 (PLSCR1) has been reported to interact with the cytoplasmic region of BACE (Kametaka et al., 2003) in a dileucine residue dependent manner, as well. Interestingly, both BACE and PLSCR1 were localized in a low buoyant lipid microdomain in SH-SY5Y cells, a potential site of interaction with APP and LRP.

LRP appears to play an important role in metabolic processing of APP. LRP mediates APP endocytosis from the cell surface (Kounnas et al., 1995; Trommsdorffet al., 1998; Ulery et al., 2000). Other reports suggest an interaction of LRP with presenilin, a member of the APP γ-secretase complex (May et al., 2002). Our current data, showing a role for LRP in trafficking of BACE, suggests that LRP has a complex role in modulating APP processing. Thus, LRP potentially acts as a scaffolding complex bringing APP, BACE, and γ-secretase together, and therefore may impact amyloid-β synthesis in Alzheimer's disease.

Interestingly, LRP also is a neuronal endocytic receptor for the Alzheimer's disease genetic risk factor apolipoprotein E, and as such acts as a clearance mechanism for amyloid-β as well (Kang et al., 2000). Thus LRP may be in a pivotal position to modulate both amyloid-β synthesis and degradation, contributing to the accumulation of amyloid-β in Alzheimer's disease.

### EXAMPLE 2

Materials and Methods

Generation of Expression Constructs of LRP and BACE

The generation of the LRP light chain with two copies of myc at its N-terminus (amino acids 3844 to 4525) (myc-LC), the mini-receptor mLRP1-myc that encodes the N-terminal cluster of ligand binding repeats fused to the light chain of LRP and tagged with myc at its C-terminus has been described previously (Mikhailenko et al., 2001). This construct was used instead of full-length LRP because of its functional similarity and better expression than full length LRP. To make mLRP1-GFP, mLRP1 was PCRed from pSecTagN2myc into pEGFP-N1 (Clontech). To create the LC-LDLR chimera, a unique KpnI restriction site was introduced into the cytoplasmic portion of LC downstream of the transmembrane domain using QuikChange XL Site-Directed Mutagenesis Kit (Stratagene, La Jolla, Calif.). Then PCR generated sequences encoding cytoplasmic domains of the human LDL receptor were inserted in place of LRP sequence. To make the LRP165-myc construct, mLRP1-myc was digested and the band containing the vector and the N-terminal 14 amino acids and the C-terminal 165 amino acids of LRP was extracted and self-ligated as described before (Kinoshita et al., 2003a). LRP light chain (amino acid 4148-4544) fused Gal4-VP16 expression construct was generated by fusing its C-terminus to the Gal4-VP 16 synthetic transcription factor, and subcloned into pcDNA3 expression vector (Invitrogen). The leader peptide from LRP was fused to HAtag epitope, which was then fused to the N-terminus of the fusion protein in the construct. The N-terminally secretory alkaline phosphatase (SEAP)-tagged LC-beta chain construct was made by inserting PCR generated sequences of SEAP and part of the LRP extracellular domain c-terminal of the furin-cleavage site, transmembrane domain and intracellular tail (amino acids 4197 to 4544) into pSeqTagB (Invitrogen); the SEAP cDNA was kindly provided by S. F. Lichtenthaler (A. Butenandt-Institut, Munich, Germany).

The Fe65-myc clone, BACE N-terminally myc tagged, C-terminally V5 tagged constructs have been described previously (Kinoshita et al., 2001; Kinoshita et al., 2003b) as well as the phosphorylation site (Walter et al., 2001) and dileucine mutants of BACE (S498D, S498A, L499A/L500A), BACE-GFP and the catalytically inactive BACE construct (D93A/D289A) (von Arnim et al., 2004). pcDNA3.1-mDab1 was a generous gift from Dr. J.

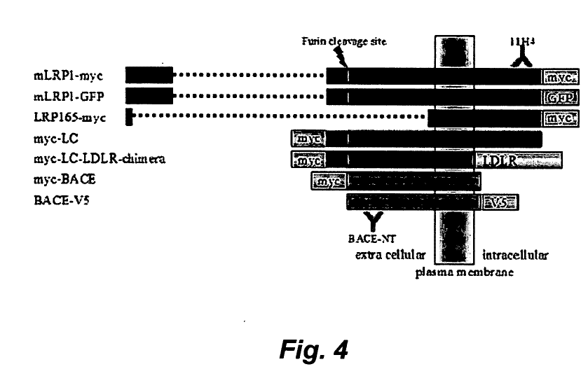

Herz (University of Texas, Dallas, Tex.). Authenticity of the PCR-generated constructs was confirmed by sequencing. The constructs used in this study are summarized in FIG. 4.

Cell Culture Conditions and Transient Transfection

H4 cells derived from human neuroglioma cells and mouse neuroblastoma N2a cells are used in this study. All cell lines were cultured in OPTI-MEMI with 10% FBS. Transient transfection of the cells was performed using a liposome-mediated method (FuGene 6; Roche Molecular Biochemicals, Indianapolis, Ind.) according to the manufacturers instruction. Cells were passaged 24 h prior to transfection and harvested or stained 24 hrs post-transfection. Primary cortical neurons were prepared as described previously (Berezovska et al., 1999).

Cortical neurons were isolated from embryonic day 16 CD1 mice (Charles River). Staining was performed 8 days after preparation.

Immunocytochemistry and Antibodies

Cells were fixed in 4% paraformaldehyde, permeabilized by 0.5% TritonX-100 in TBS and blocked with 1.5% normal goat serum. For surface staining Triton-X treatment was omitted. The following antibodies were used: the Golgi organelle marker GM130 mAb and the FITC-conjugated endosomal marker EEA mAb (BD Transduction Laboratories, San Diego, Calif.). The tag antibodies used were rabbit anti-myc Ab (Upstate Biotechnology, Lake Placid, N.Y.), anti-myc mAb, anti-V5 mAb (both from Invitrogen, Carlsbad, Calif.) and rabbit anti-V5 Ab (Abcam, Cambridge, Mass.). Antibodies raised in rabbit against the N-(46-56) and C-termini (487-501) of BACE were obtained from Calbiochem (San Diego, Calif.). A hybridoma secreting an mAb to the LRP intracellular domain (11H4) was obtained from the American Type Culture Collection (Rockville, Md.). Alexa-555-labeled cholera toxin B (CTx-B, Molecular Probes Inc., Eugene, Oreg.) was used to visualize lipid rafts. Secondary antibodies used were labeled with FITC and Cy3 (Jackson Immunoresearch, West Grove, Pa.) or Alexa 488 (Molecular Probes). Immunostained cells were coverslipped and mounted for confocal or two photon microscopic imaging. The immunostained cells were observed with the appropriate filters by confocal microscopy using a BioRad 1024 confocal 3-channel instrument.

Cholesterol Depletion

For cholesterol depletion, H4 cells were grown for 24 h in Opti-MEM with 10% FBS and then for 24 h in either DMEM supplemented with 2 mM L-glutamine, 10% delipidated FBS (Cocalico Biologicals), 20 μM lovastatin (Calbiochem) and 0.5 mM mevalonate (mevalonolactone, Sigma) or DMEM with 10% complete FBS (Liscum et al., 2002). Prior to experimentation, cells were incubated for 10 min in DMEM with 10 mM methyl-β-cyclodextrin (MP-CD, Sigma) or DMEM alone.

Co-Immunoprecipitation

Immunoprecipitation experiments were carried out with BioMag goat anti-mouse IgG (PerSeptive Biosystems, Framingham, Mass.). The magnetic beads were incubated overnight at 4° C. with anti-V5 or anti-myc mAb or TBS alone. Lysates from H4 cells cotransfected with BACE-V5 and mLRP1-myc or pure lysis buffer were added to the bead-antibody complex for 2 h at 4° C. After the supernatants were collected, the beads were washed in lysis buffer and then boiled with 2× Tris-glycine SDS sample buffer (Invitrogen) for 3 mins. The supernatants were loaded onto 10-20% Tris-glycine polyacrylamide gels (Novex, San Diego, Calif.) under denaturing and reducing conditions. The proteins were transferred to immobilon-P membrane (Millipore, Bedford, Mass.) and blocked in 5% non-fat dried milk. mLRP1-myc was detected by rabbit anti-myc Ab. BACE was detected by rabbit anti-BACE-NT Ab. Secondary antibodies conjugated to horseradish peroxidase were applied and visualized by chemiluminescence. The Massachusetts Alzheimer Disease Research Center Brain Bank provided temporal cortex. Our protein solubilization procedure was adapted from previously reported studies (Orlando et al., 2002) with minor modifications. The tissue was homogenized at 1 mL/100 mg tissue in ice-cold TEVP-sucrose buffer containing 10 mM Tris, pH 7.4, 5 mM NaF, 1 mM Na₃VO₄, 1 mM EDTA, 1 mM EGTA, and 320 mM sucrose. The homogenates were centrifuged at 4° C., and the supernatants were removed. The pellets were resuspended in 800 uL TEVP with 1% SDS, sonicated for 10 s, and then boiled for 5 min. The samples were centrifuged and the supernatant was collected for IP after the protein concentration was determined by protein assay (BioRad). Co-IP in human brain tissue was performed as described above with rabbit anti-BACE-CT as pull-down Ab and probed with 11H4 mAb.

FRET Measurements using Fluorescence Lifetime Imaging Microscopy (FLIM)

FRET is observed when two fluorophores are in very close proximity, i.e., <10 nm. FRET measurements using FLIM relies on the observation that fluorescence lifetimes (the time of fluorophore emission after brief excitation, measured in picosec) are shorter in the presence of a FRET acceptor. We have utilized a new FLIM technique that can detect protein-protein proximity using multiphoton microscopy (Bacskai et al., 2003; Berezovska et al., 2003). A mode-locked Ti-sapphire laser (Spectra Physics) sends a ˜100 fsec pulse every ˜12.5 nsec to excite the fluorophore. Images were acquired using a BioRad Radiance 2000 multiphoton microscope. We used a high-speed Hamamatsu MCP detector (MCP5900; Hamamatsu, Ichinocho, Japan) and hardware/software from Becker and Hickl (SPC 830, Berlin, Germany) to measure fluorescence lifetimes on a pixel-by-pixel basis. Excitation at 800 nm was empirically determined to excite GFP, Alexa488 and FITC, but not Cy3. Donor fluorophore (GFP, Alexa488 or FITC) lifetimes were fit to two exponential decay curves to calculate the fraction of fluorophores within each pixel that either interact, or do not interact, with an acceptor. As a negative control, GFP, Alexa488 or FITC lifetimes were measured in the absence of acceptor (Cy3), which showed lifetimes equivalent to GFP, Alexa488-IgG or FITC IgG alone, in solution or with cotransfection with an empty vector (pEGFP) measured in the presence of Cy3 labeled BACE-V5 or LC-myc. No bleedthrough or mis-excitation of Cy3 was observed under these conditions. Statistical testing was performed by Student's t-test.

Internalization Assay

To quantitate BACE internalization we employed a modified protocol by Sever et al. (2000). CHO 13-5-1 (LRP null cells) were grown to 70% confluency in 6-well plates and transiently transfected with myc-BACE and either empty-vector or LC-GFP. Cells were then washed once with ice-cold PBS containing 1 mM CaCl₂ and 1 mM MgCl2, 0.2% BSA and 5 mM glucose (Happy Buffer) and 0.4 μg/ml biotinylated myc-mAb (Upstate) in Happy Buffer was applied for 30 min on ice. After that the cells were allowed to endocytose at 37° C. for the indicated times. Returning the plates on ice stopped endocytosis. Surface biotin was masked with streptavidin (Roche) for 1 h on ice. Avidin was quenched with 0.5 mg/ml biocytin (Sigma). Cells were harvested in blocking buffer (1% Triton-X-100, 0.1% SDS, 0.2% BSA, 50 mM NaCl, 1 mM Tris [pH7.4]) and incubated on IgG coated 96-well plates at 4° C. overnight. Then the plates underwent a wash cycle of 3× in PBS, and were incubated in streptavidin-POD 1:5000 (Roche) in blocking buffer for 1 h. After another wash cycle of 3× in PBS the plates were incubated with 200 μl of 10 mg o-phenyldiamine HCl (Sigma), 10 μl 30% H₂O₂ (Sigma) in 25 ml of 50 mM Na2HPO4, 27 mM citrate (pH5.0). The reaction was terminated by the addition of 50 μl H2SO4 and the A490 was read. BACE internalization was then graphed as percentage of internalized myc-BACE of total surface myc-BACE.

Western Blotting

N2a cells cotransfected with LC-myc and either empty vector, BACE, or a catalytically inactive BACE mutant were lysed in 1% Triton-X in TBS buffer and proteinase inhibitor tablets (Roche) and then loaded onto 10-20% Tris-glycine polyacrylamide gels (Novex, San Diego, Calif.) under denaturing and reducing conditions. The proteins were transferred to immobilon-P membrane (Millipore, Bedford, Mass.). Staining was performed according to Licor odyssey instructions and LRP light chain was detected by rabbit anti-myc Ab with 680 gt anti rabbit secondary and visualized on the Licor odyssey.

Phosphatase Assay

N2a cells passaged into 12-well plates were transfected with a β-galactosidase (β-gal) reporter, LRPβ fused N-terminally to secreted alkaline phosphatase and either empty vector, BACE, or a catalytically inactive BACE mutant. Each condition was transfected in triplicate. Media was changed 24 hours later, and then collected after another 24 hours. Measurement of SEAP activity in the conditioned media was carried out in triplicate by chemiluminescent assay (Roche, Mannheim, Germany) according to the manufacturers instructions. SEAP activity was normalized to β-gal activity, which was measured by hydrolysis of o-nitrophenyl-β-D-galactopyranoside in cells lysed with reporter lysis buffer (Promega, Madison, Wis.).

Luciferase Assay

HEK 293 cells were transfected with LRP-Gal4-VP 16 (LRP-GV) in the absence or presence of BACE and relative luciferase activity determined (May et al., 2002). Activity relative to β galactosidase is shown; average of triplicate analyses. In all cases transfection was confirmed by immunoblotting.

Results

Localization of BACE and LRP-Constructs

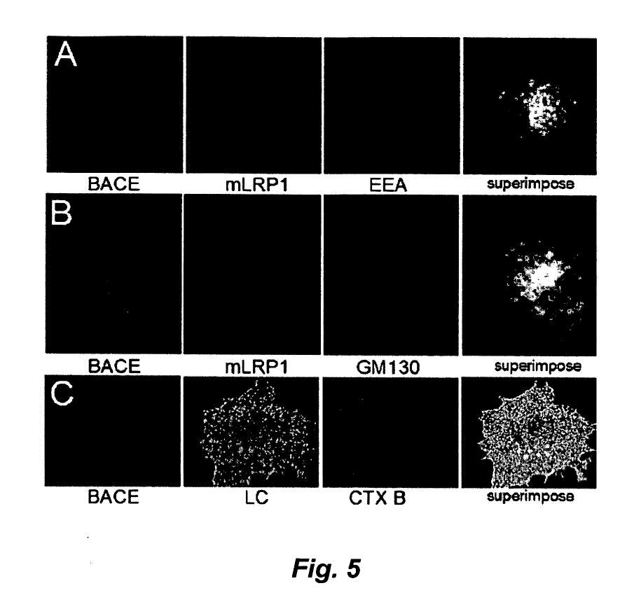

We first tested the localization of BACE and LRP-constructs in co-expressing H4 cells. When expressed individually, both mLRP1 and BACE were localized mainly in punctate structures in the cells; mLRP1-positive structures largely overlapped with BACE-positive structures when they were co-expressed. To determine the subcellular distribution we immunostained co-expressing H4 cells with organelle markers or, in cells surface staining without Triton-X treatment, Alexa555 labeled CTx-B as a raft marker. mLRP1 and BACE co-localized in the endosomal compartments stained by EEA (FIG. 5A). To a lesser extent, the Golgi marker GM130 also overlapped with mLRP1 (FIG. 5B). On the cell surface myc-LC and BACE are partly colocalized with lipid rafts. The results of the immunocytochemistry suggest that LC and BACE are co-localized in distinct compartments of the cell including lipid rafts, Golgi and prominently in the endosomal compartment.

Co-Immunoprecipitation of BACE and LRP

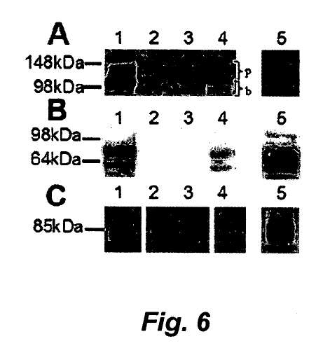

From the immunohistochemical experiments that showed robust co-localization, we hypothesized that there may be a close interaction between LRP and BACE. To test whether LRP interacts with BACE, we immunoprecipitated BACE from cotransfected H4 cells (FIG. 6) and probed for mLRP1 (lane 1), controlling for non-specific interactions by assessing lysates incubated without the pull-down antibody (lane 2) or pure lysis buffer (lane 4). Immunoreactive bands of approx. 100 kDa (resembling mature furin-cleaved mLRP1-myc) and approx. 140 kDa (resembling unprocessed Golgi and ER forms of mLRP1-myc) (Mikhailenko et al., 2001) were detected in the immunoprecipitated sample and the whole-cell lysate (lane 4, FIG. 6A). In order to confirm this interaction, the complementary pull down experiment was performed. mLRP1 immunoprecipitates were probed by an anti-BACE Ab with the same controls. The doublet bands of approx. 60 kDa and 75 kDa were detected only in lane 1 and in the control lysate lane (FIG. 6B), suggesting that BACE is present in the mLRP1 immunoprecipitates. The 60 kDa band represents endogenous BACE, whereas the 75 kDa band represents transfected BACE containing V5-His tag. To demonstrate a direct interaction of LRP and BACE under physiological conditions in brain, where BACE function is presumed to be important in the pathogenesis of Alzheimer's disease, we immunoprecipitated BACE from human brain tissue and probed with an antibody to the LRP light chain. A strong immunoreactive band of approx. 85 kDa (the expected size of the mature endogenous light chain of LRP, i.e. furin cleaved form) (Mikhailenko et al., 2001) was detected in the sample lane (lane 1, FIG. 6C). Control precipitate from samples lacking anti-BACE Ab (lane 2) showed only a weak immunoreactive band and samples lacking cell extract (lane 3) did not contain this band. Thus, endogenous LRP and BACE co-immunoprecipitate.

Interaction of LRP and BACE by FLIM analysis

We next used an alternative technique to probe protein-protein proximity to test the idea that the LRP-BACE interaction detected by co-IP occurs in specific cell compartments, and to further evaluate the biochemical parameters of this interaction. We utilized fluorescence lifetime imaging microscopy (FLIM), a morphology based FRET technique that can reveal close protein-protein proximity in intact cells. Fluorescence lifetime is influenced by the surrounding microenvironment and is shortened in the immediate vicinity of a FRET acceptor molecule. The degree of lifetime shortening can be displayed with very high spatial resolution in a pseudo-color-coded image. As shown in FIG. 5, double immunostaining showed subcellular compartment colocalization of BACE and LRP predominantly in endosomal compartments, but this does not necessarily imply a close interaction. We measured changes in the lifetime of the donor fluorophore (either FITC, Alexa488 or GFP) under different experimental conditions. In the absence of an acceptor fluorophore, the lifetime of FITC conjugated to IgG (hereafter referred to simply as FITC) is ˜2300 psec, GFP ˜2200 psec and Alexa488 is 1900 psec. If an acceptor fluorophore is present but remains too distant from the donor (i.e. there is no interaction), donor lifetimes remain in this range. The lifetime of FITC attached to the C-terminus of BACE-V5 alone (2334+/−72 psec) was significantly shortened when coexpressed mLRP1-myc was C-terminally labeled by Cy3 (2153+/−55 psec, p<0.0001), indicating FRET between the two fluorophores (Table 3). Equivalent results were obtained when BACE was tagged with GFP and when the acceptor and donor fluorophores were exchanged (Table 3).

TABLE 3
FLIM assay data for proximity of various LRP-constructs and BACE in transfected H4 cells

| | | Lifetime | | |
|---|---|---|---|---|
| | | (picosec) | | Significance |
| Donor | Acceptor | (Mean +/− SD) | n | (compared to control) |
| BACE-V5 (FITC) | none | 2334 +/− 72 | 12 | — |
| BACE-V5 (FITC) | mLRP1-myc (Cy3) | 2153 +/− 55 | 11 | p < 0.0001 |
| mLRP1-myc (FITC) | none | 2260 +/− 109 | 17 | — |
| mLRP1-myc (FITC) | BACE-V5 (Cy3) | 1703 +/− 401 | 15 | p < 0.0001 |
| mLRP1-GFP | none | 2164 +/− 10 | 5 | — |
| mLRP1-GFP | BACE-V5 (Cy3) | 1165 +/− 8 | 5 | p < 0.0001 |
| mLRP1-GFP | myc-BACE (Cy3) | 2247 +/− 102 | 5 | n.s. |
| BACE-GFP | none | 2284 +/− 20 | 6 | — |
| BACE-GFP | mLRP1-myc (Cy3) | 1756 +/− 50 | 4 | p = 0.0004 |
| pEGFP-N1-vector | none | 2157 +/− 71 | 5 | — |
| pEGFP-N1-vector | BACE-V5 (Cy3) | 2106 +/− 37 | 5 | n.s. |
| pEGFP-N1-vector | mLRP1-myc (Cy3) | 2138 +/− 101 | 5 | n.s. |
| Myc-LC (11H4-FITC) | none | 2305 +/− 69 | 15 | — |
| Myc-LC (11H4-FITC) | BACE-V5 (Cy3) | 2100 +/− 148 | 15 | p < 0.0001 |
| LRP165-myc (11H4-FITC) | none | 2316 +/− 86 | 13 | — |
| LRP165-myc (11H4-FITC) | BACE-V5 (Cy3) | 1803 +/− 283 | 16 | p < 0.0001 |

FLIM analysis data for proximity of various LRP and BACE-constructs within transfected H4 cells. If there is no interaction, lifetimes of the donor fluorophore are similar to the negative control (lacking the acceptor fluorophore). Statistically shorter lifetimes between BACE and mLRP1 regardless of the combination of fluorophore at their C-terminus indicate FRET between them. To further determine the interaction site of BACE and LRP, FLIM between deletion constructs was performed.

In order to test the idea that the decrease in lifetime observed in the mLRP1-GFP/BACE-V5 FLIM assay was due to FRET, we performed an additional control. mLRP1-GFP and myc-BACE co-transfected cells were stained with anti-myc Ab, then labeled with Cy3. In this experiment, the myc tag was at the N-terminus of BACE, across the membrane (and, therefore, too distant from the C-terminal GFP on mLRP1-GFP to be detected by FRET). Although there was striking colocalization, no lifetime change was observed. This experiment demonstrates the specificity of the proximity assay in this FLIM-based method of measuring FRET.

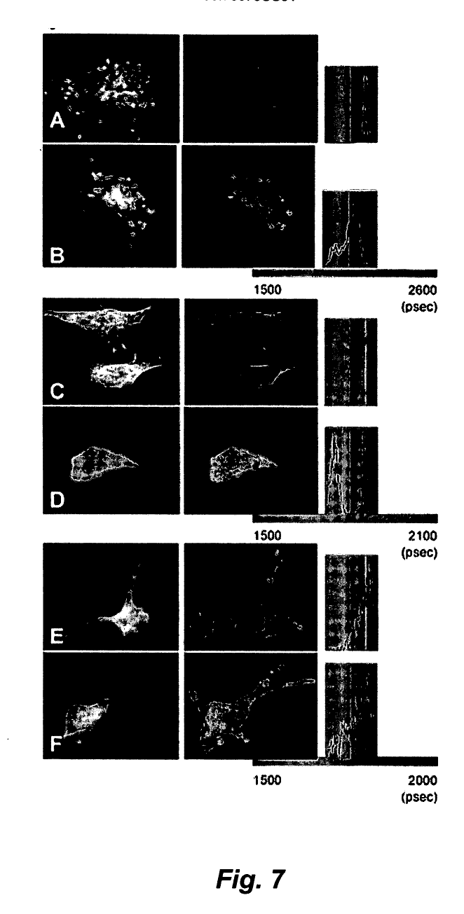

FLIM allows analysis of FRET localization by recording the distribution of donor lifetimes on a pixel-by-pixel basis. FITC bound to BACE-V5 in the absence of acceptor has a uniform lifetime and a single lifetime peak (FIG. 7A); in the presence of mLRP1-Cy3 acceptor, FITC has two distinct lifetimes and is faster, representing “FRETting” molecules (FIG. 7B). Examination of the FLIM images of transfected cells suggests that LRP and BACE interact (red pseudocolor) at the cell surface and in endosomal compartments. Since LRP and BACE colocalize in lipid rafts at the cell surface, we suggest that the interaction detected by FLIM also is lipid raft associated.

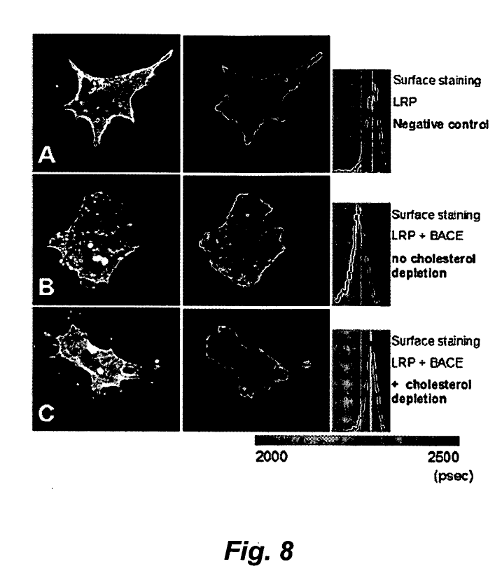

Indeed, additional experiments in which only cell surface myc-LC (N-terminal myc) and BACE-NT are immunostained demonstrate FRET (Table 4) between BACE and LRP specifically in punctuate structures at the cell surface (FIG. 8B). To test the hypothesis that this interaction occurs in rafts we cholesterol depleted the cells and repeated the cell surface FLIM experiment. Cholesterol depletion weakened the interaction of BACE and LRP at the cell surface (FIG. 8C). This result strongly suggests that BACE and LRP interact on the cell surface distinctively in lipid rafts.

TABLE 4
FLIM assay data showing the proximity of LC and BACE and strong weakening of this
proximity by cholesterol depletion (chol. depl.) and substitution of the LRP intracellular
domain by LDLR in cell surface staining.

| | | | Lifetime | | Significance | | | | |
|---|---|---|---|---|---|---|---|---|---|
| | | Chol. | (picosec) | | (compared | | | | |
| Donor | Acceptor | Depl. | (Mean +/− SD) | n | to control) | | | | |
| Myc-LC (FITC) | none | − | 2317 +/− 109 | 21 | — | | | | |
| Myc-LC (FITC) | BACE-NT | − | 2023 +/− 173 | 23 | p < 0.0001 | | | | |
| | (Cy3) | | | | | {close oversize bracket} | p < 0.0001 | | |
| Myc-LC (FITC) | BACE-NT (Cy3) | + | 2203 +/− 83 | 21 | p < 0.001 | | | {close oversize bracket} | p < 0.005 |
| Myc-LC-LDLR | none | − | 2335 +/− 111 | 8 | — | | | | |
| Myc-LC-LDLR | BACE-NT (Cy3) | − | 2243 +/− 143 | 8 | n.s. | | | | |

FLIM analysis data of LRP-BACE proximity on the cell surface in the presence or absence of lipid rafts. H4 cells were co-transfected with myc-(N-terminus)-LC or an LRP-LDLR chimera (labeled by FITC) and BACE-V5 (labeled by Cy3). Cholesterol depletion was performed with lovastatin/mevalonate for 24 h and MβCD for 10 min.

To further confirm the physiologic interaction of LRP and BACE we performed FLIM in untransfected N2a cells as well as primary cortical neurons, which have relatively abundant BACE and LRP; Alexa488 was chosen as donor fluorophore because it is somewhat brighter than FITC under the conditions utilized. Statistically shorter lifetimes of the donor in N2a cells (Alexa488) attached to the C-terminus of BACE in the presence of C-terminally Cy3-labeled LRP (1492+/−293 psec, p<0.0001) compared to only Alexa488-labeled BACE (1868+/−116 psec) as well as in FITC-labeled primary neurons attached to the C-terminus of BACE in the presence of C-terminally Cy3-labeled LRP (2055+/−103 psec, p≦0.005) compared to only FITC-labeled BACE (2193+/−105 psec) indicate FRET between endogenously expressed proteins (Table 5). In the absence of acceptor, Alexa488 has a uniform lifetime; in the presence of acceptor a second peak appears, reflecting an interaction (FIGS. 7C-F). This interaction, pseudocolored red, also appears to be stronger in the distal compartments at or near the cell surface. This result demonstrates close protein-protein interaction between endogenous LRP and endogenous BACE at the cell surface in a neuronal cell type, paralleling the co-IP results.

TABLE 5
FLIM assay data showing the proximity of endogenous LRP and
BACE in N2a cells and in primary neurons.

| | | | Lifetime | | Significance |
|---|---|---|---|---|---|
| | | | (picosec) | | (compared to |
| | Donor | Acceptor | (Mean +/− SD) | n | control) |
| N2a cells | BACE-CT | none | 1868 +/− 116 | 20 | — |
| | (Alexa 488) | | | | |
| | BACE-CT | 11H4 | 1492 +/− 293 | 20 | p < 0.0001 |
| | (Alexa 488) | (Cy3) | | | |
| Primary | BACE-CT | none | 2193 +/− 105 | 15 | — |
| neurons | (FITC) | | | | |
| | BACE-CT | 11H4 | 2055 +/− 108 | 10 | p ≦ 0.005 |
| | (FITC) | (Cy3) | | | |

FLIM analysis data for endogenous LRP and BACE. N2a cells were stained for endogenous BACE with rabbit anti-BACE-CT Ab (labeled by Alexa488) and LRP with 11H4 (labeled by Cy3).

To identify the domain of LRP interacting with BACE, we utilized LRP deletion constructs. LC and LRP165—a construct that contains only the 100 amino acid intracellular domain, the transmembrane domain and a very small extension beyond the membrane—both interacted strongly with BACE (Table 3). This result implicates the intracellular or the transmembrane domain of LRP as the site of interaction. To further test this hypothesis, we utilized a chimeric protein in which the extracellular and transmembrane domains of LC are fused to the intracellular domain of the low-density lipoprotein receptor (FIG. 4) and performed FLIM on the cell surface as described above. This construct did not FRET with BACE (Table 4), further supporting the importance of the intracellular domain of LRP for this interaction.

There are several scaffold/adaptor proteins known to interact with LRP including Fe65 (Trommsdorff et al., 1998; Kinoshita et al., 2001; Pietrzik et al., 2004) and mammalian disabled 1 (mDabl) (Trommsdorff et al., 1998). If the intracellular domain is responsible for the interaction between LRP and BACE, these adaptor proteins may play a role in the interaction. We have demonstrated previously that LRP and Fe65 interact using a FRET based cell assay (Kinoshita et al., 2001), and that Fe65 is responsible for mediating an LRP-Fe65-APP heterotrimeric complex. We therefore examined the possibility that these molecules may interact with BACE by the FRET assay. However, under the conditions utilized we did not detect any FRET between BACE and Fe65 or between BACE and mDabl (data not shown). Recent data also suggest that phosphorylation of BACE at Ser498 changes its trafficking possibly by altering its interactions with GGA by its C-terminal dileucine-motif (He et al., 2002; von Arnim et al., 2004). We also generated the S498D, S498A and L499A/L500A mutants of BACE to evaluate if these mutants, which mimic or block phosphorylation of Ser498 (Walter et al., 2001), alter interaction with LRP. No change in FRET measures were observed with these manipulations (data not shown).

BACE Internalization Assay

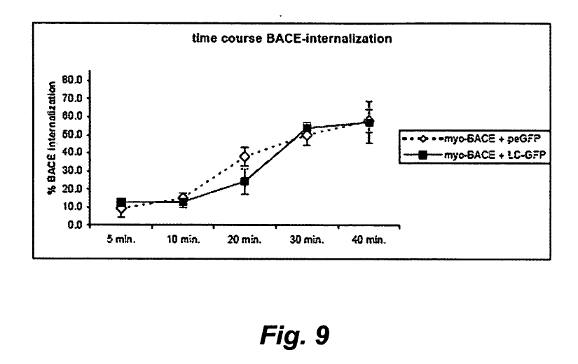

In order to assess the effect of LRP on BACE endocytosis we assessed internalization of biotinylated n-terminally myc-tagged BACE and assayed internalized versus cell-surface BACE over time (FIG. 9). Co-transfection with LC did not enhance BACE internalization from the cell surface in LRP null CHO cells (13-5-1) in contrast to known enhancement of APP-endocytosis with LC (Pietrzik et al., 2002).

LRP Shedding

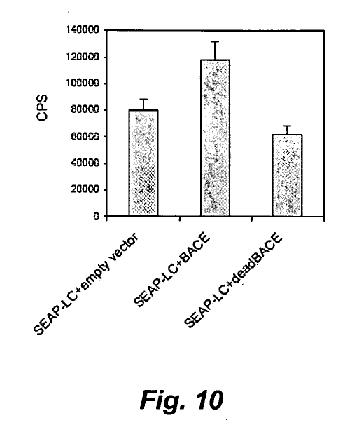

In order to assess the effect of BACE and LRP interaction on LRP processing, we measured shedding of the LRP with the cDNA of secreted alkaline phosphatase (SEAP) fused to the N-terminus of the LRP beta-chain. An analogous study has been described to study BACE cleavage of APP (Lichtenthaler et al., 2003). After cotransfection of the SEAP-LRP construct with a β-gal reporter construct and either empty vector, WT-BACE or BACE D93/289A, SEAP activity was measured in the media and normalized to β-gal activity. WT-BACE led to a significant increase in LRP cleavage compared to baseline, while catalytically inactive BACE D93/289A exhibited no effect as expected (FIG. 10).

LRP C-Terminal Fragment (CTF) Production after BACE Cotransfection

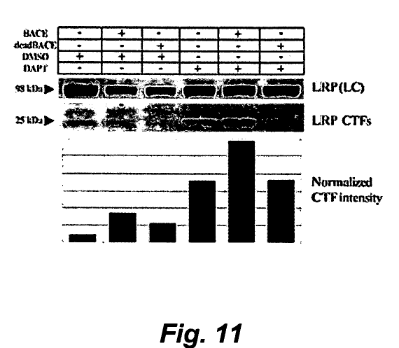

After treatment with DAPT for 12 h we observed an increase of the LRP-CTF 25 kDa band consistent with the observations of (May et al., 2002) suggesting gamma cleavage of LRP. After cotransfection of LRP with BACE we saw an increase of LRP-CTF as well as in non-treated cells and even more after DAPT treatment. Cotransfection with a catalytically inactive BACE mutant blocked the increase in LRP-CTF generation (FIG. 11). These results are consistent with a BACE-mediated cleavage of LRP, generating a CTF for gamma cleavage.

Luciferase Assay

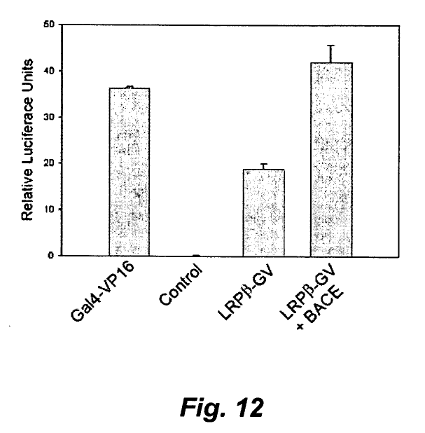

If LRP interacts with the β secretase it may undergo cleavage and generate a truncated form. By analogy to APP, it may then undergo further proteolysis and release the LRP-ICD fragment. Evidence that LRP is itself a y substrate has been presented using an LRP-Gal4/VP 16 construct utilizing a luciferase reporter assay for LRP C-terminal cleavage (May et al., 2002). We used this same assay to determine if co-transfection with BACE would alter generation of this putative signaling domain. Cotransfection with BACE led to a substantial increase in luciferase activity suggesting that LRP undergoes BACE cleavage, leading ultimately to release of the cytoplasmic domain and translocation to the nucleus to activate the Gal4 assay (FIG. 12).

Discussion

In the present study, we demonstrate that BACE interacts with the intracellular domain of LRP, a multi-functional endocytic receptor. The interaction is demonstrated by co-IP of BACE and LRP from over-expressing cells and from endogenous BACE and LRP in human brain samples and primary neurons. Co-localization and close proximity in both H4 and N2a cells is shown by confocal microscopy and FRET-based proximity assays, suggesting co-localization primarily in endosomes and at the cell surface. Cholesterol depletion disrupted the cell surface LRP-interaction, suggesting that the interaction occurs in lipid rafts.

The FRET technique used here, FLIM, is advantageous because it provides quantitative data on protein-protein proximity with exquisite subcellular localization. The two fluorophores must be quite close (<10 nm) to support FRET; tagging LRP and BACE molecules “across the membrane” from one another or after cholesterol depletion abolishes FRET despite continued co-localization at the light level. Analogous results were obtained using three different fluorophores, multiple different antibody pairs, endogenous or transfected LRP and BACE, and two different cell types. Taken together with the co-IP data, our results strongly support the conclusion that LRP and BACE interact at the cell surface in raft compartments.

BACE interacts with an increasing number of other proteins. Sialyltransferase and P-selectin glycoprotein ligand-1 (PSGL-1) are reported to be substrates of BACE (Kitazume et al., 2001; Lichtenthaler et al., 2003). Nicastrin (Hattori et al., 2002) is found to activate BACE in the APP, BACE, PS 1 and nicastrin complex. A family of GGA adapter proteins and the phospholipid scramblase 1 (PLSCR1) have been shown to directly interact with the BACE tail (He et al., 2002; Kametaka et al., 2003). Interestingly, both BACE and PLSCR1 were localized in a low buoyant lipid microdomain, a potential site of interaction with APP and LRP.

Our data address the subcellular localization of BACE and LRP interactions. Although BACE and LRP are mostly colocalized in the endosomal compartments and to a lesser extent in the Golgi and on the cell surface as shown by conventional immunostaining, our FLIM data suggest that they come into closest proximity at the cell surface in lipid rafts, where it has been shown that amyloidogenic processing seems to occur in raft associated compartments (Ehehalt et al., 2003). This is in good accordance with a recent paper showing that ApoE, a LRP ligand, is colocalized with APP and BACE in lipid rafts (Kawarabayashi et al., 2004). Since even the LRP165-construct (which lacks ligand binding domains and most of the extracellular part of LRP) interacts with the BACE intracellular domain and the LC-LDLR-chimera lacking the LRP intracellular domain did not interact with BACE, we postulate that the major interaction site is the intracellular domain of LRP. While two candidate adaptor proteins, Fe65 and mDab, did not appear to mediate the interaction, multiple other potential adaptor proteins might either mediate or impact this interaction. Surprisingly we did not find enhancement of BACE endocytosis in LRP null CHO (13-5-1) cells cotransfected with LRP. Our current data, showing an interaction between LRP and BACE, suggest that LRP has a complex role in modulating APP processing. Thus, LRP, which is highly enriched in rafts, potentially acts as a scaffolding complex bringing APP and BACE together; such an interaction may help explain the observations that directing APP to rafts enhances β cleavage and Aβ generation (Ehehalt et al., 2003). Interestingly, LRP also is a neuronal endocytic receptor for the Alzheimer's disease genetic risk factor apolipoprotein E, and as such acts as a clearance mechanism for Aβ as well (Kang et al., 2000). However it is possible that LRP competes with APP for BACE cleavage. Thus LRP may be in a pivotal position to modulate both Aβ synthesis and degradation, and may thus have a role in the accumulation of Aβ in Alzheimer's disease.

Additionally we have shown that BACE activity leads to an increase of secreted LRP in the media as well as LRP-CTF, in analogy to APP processing. Moreover BACE overexpression leads to an increase of γ-secretase like cleavage to release LRP-ICD. Thus APP and LRP directly and indirectly interact with one another and co-traffic to the cell surface. Surprisingly we now show that LRP is a substrate for both BACE and γ-secretase, identified as APP processing enzymes. Whether this processing leads to a stable LRP equivalent of Aβ is unknown. Moreover, unlike Notch processing, so far neither our studies nor those of May et al. (2002) suggest a strong effect of ligand (e.g. α2macroglobulin) binding on secretase activity on LRP. However, both LRP (Bacskai et al., 2000; Boucher et al., 2002) and other members of the LDL receptor related family (Trommsdorff et al., 1999) have been implicated as having signaling roles in neurons, and it seems likely that the cleavage of LRP we observe could modulate such signaling. Further studies will be needed to elucidate if APP and LRP act co-operatively or competitively for access to these secretases and how interactions of LRP with other ligands impact these processes.

### REFERENCES

Bacskai B J, Xia M Q, Strickland D K, Rebeck G W, Hyman B T (2000) The endocytic receptor protein LRP also mediates neuronal calcium signaling via N-methyl-D aspartate receptors. Proc Natl Acad Sci USA 97:11551-11556.
Bacskai B J, Skoch J, Hickey G A, Allen R, Hyman B T (2003) Fluorescence resonance energy transfer determinations using multiphoton fluorescence lifetime imaging microscopy to characterize amyloid-beta plaques. J Biomed Opt 8:368-375.
Bennett B D, Denis P, Haniu M, Teplow D B, Kahn S, Louis J C, Citron M, Vassar R (2000) A furin-like convertase mediates propeptide cleavage of BACE, the Alzheimer's beta-secretase. J Biol Chem 275:37712-37717.
Berezovska 0, Rarndya P, Skoch J, Wolfe M S, Bacskai B J, Hyman B T (2003) Amyloid precursor protein associates with a nicastrin-dependent docking site on the presenilin 1-gamma-secretase complex in cells demonstrated by fluorescence lifetime imaging. J Neurosci 23:4560-4566.
Berezovska O, Frosch M, McLean P, Knowles R, Koo E, Kang D, Shen J, Lu F M, Lux S E, Tonegawa S, Hyman B T (1999) The Alzheimer-related gene presenilin 1 facilitates notch 1 in primary mammalian neurons. Brain Res Mol Brain Res 69:273-280.
Boucher P, Liu P, Gotthardt M, Hiesberger T, Anderson R G, Herz J (2002) Platelet derived growth factor mediates tyrosine phosphorylation of the cytoplasmic domain of the low Density lipoprotein receptor-related protein in caveolae. J Biol Chem 277:15507-15513.
Capell A, Steiner H, Willem M, Kaiser H, Meyer C, Walter J, Lammich S, Multhaup G, Haass C (2000) Maturation and pro-peptide cleavage of beta-secretase. J Biol Chem 275:30849-30854.
Cordy J M, Hussain I, Dingwall C, Hooper N M, Turner A J (2003) Exclusively targeting beta-secretase to lipid rafts by GPI-anchor addition up-regulates beta-site processing of the amyloid precursor protein. Proc Natl Acad Sci USA 100:11735-11740.
Creemers J W, Ines Dominguez D, Plets E, Semeels L, Taylor N A, Multhaup G, Craessaerts K, Annaert W, De Strooper B (2001) Processing of beta-secretase by furin and other members of the proprotein convertase family. J Biol Chem 276:4211-4217.
Ehehalt R, Keller P, Haass C, Thiele C, Simons K (2003) Amyloidogenic processing of the Alzheimer beta-amyloid precursor protein depends on lipid rafts. J Cell Biol 160:113-123.
Hattori C, Asai M, Oma Y, Kino Y, Sasagawa N, Saido T C, Maruyama K, Ishiura S (2002) BACE1 interacts with nicastrin. Biochem Biophys Res Commun 293:1228-1232.
He X, Chang W P, Koelsch G, Tang J (2002) Memapsin 2 (beta-secretase) cytosolic domain binds to the VHS domains of GGA1 and GGA2: implications on the endocytosis mechanism of memapsin 2. FEBS Lett 524:183-187.
Herz J, Strickland D K (2001) LRP: a multifunctional scavenger and signaling receptor. J Clin Invest 108:779-784.
Higashi S, Miyazaki K (2003) Novel processing of beta-amyloid precursor protein catalyzed by membrane type 1 matrix metalloproteinase releases a fragment lacking the inhibitor domain against gelatinase A. Biochemistry 42:6514-6526.
Huse J T, Pijak D S, Leslie G J, Lee V M, Doms R W (2000) Maturation and endosomal targeting of beta-site amyloid precursor protein-cleaving enzyme. The Alzheimer's disease beta-secretase. J Biol Chem 275:33729-33737.
Hussain I, Powell D, Howlett D R, Tew D G, Meek T D, Chapman C, Gloger I S, Murphy K E, Southan C D, Ryan D M, Smith T S, Simmons D L, Walsh F S, Dingwall C, Christie G (1999) Identification of a novel aspartic protease (Asp 2) as beta secretase. Mol Cell Neurosci 14:419-427.
Kametaka S, Shibata M, Moroe K, Kanamori S, Ohsawa Y, Waguri S, Sims P J, Emoto K, Umeda M, Uchiyama Y (2003) Identification of phospholipid scramblase 1 as a novel interacting molecule with beta-secretase (beta-site amyloid precursor protein (APP) cleaving enzyme (BACE)). J Biol Chem 278:15239-15245.
Kang D E, Pietrzik C U, Baum L, Chevallier N, Merriam D E, Kounnas M Z, Wagner S L, Troncoso J C, Kawas C H, Katzman R, Koo E H (2000) Modulation of amyloid beta-protein clearance and Alzheimer's disease susceptibility by the LDL receptorrelated protein pathway. J Clin Invest 106:1159-1166.
Kawarabayashi T, Shoji M, Younkin L H, Wen-Lang L, Dickson D W, Murakami T, Matsubara E, Abe K, Ashe K H, Younkin S G (2004) Dimeric amyloid beta protein rapidly accumulates in lipid rafts followed by apolipoprotein E and phosphorylated tau accumulation in the Tg2576 mouse model of Alzheimer's disease. J Neurosci 24:3801-3809.
Kinoshita A, Shah T, Tangredi M M, Strickland D K, Hyman B T (2003a) The intracellular domain of the low density lipoprotein receptor-related protein modulates transactivation mediated by amyloid precursor protein and Fe65. J Biol Chem 278:41182-41188.
Kinoshita A, Fukumoto H, Shah T, Whelan C M, Irizarry M C, Hyman B T (2003b) Demonstration by FRET of BACE interaction with the amyloid precursor protein at the cell surface and in early endosomes. J Cell Sci 116:3339-3346.
Kinoshita A, Whelan C M, Smith C J, Mikhailenko I, Rebeck G W, Strickland D K, Hyman B T (2001) Demonstration by fluorescence resonance energy transfer of two sites of interaction between the low-density lipoprotein receptor-related protein and the amyloid precursor protein: role of the intracellular adapter protein Fe65. J Neurosci 21:8354-8361.
Kitazume S, Tachida Y, Oka R, Shirotani K, Saido T C, Hashimoto Y (2001) Alzheimer's beta-secretase, beta-site amyloid precursor protein-cleaving enzyme, is responsible for cleavage secretion of a Golgi-resident sialyltransferase. Proc Natl Acad Sci USA 98:13554-13559.
Kitazume S, Tachida Y, Oka R, Kotani N, Ogawa K, Suzuki M, Dohmae N, Takio K, Saido T C, Hashimoto Y (2003) Characterization of alpha 2,6-sialyltransferase cleavage by Alzheimer's beta-secretase (BACE1). J Biol Chem 278:14865-14871.
Kounnas M Z, Moir R D, Rebeck G W, Bush A I, Argraves W S, Tanzi R E, Hyman B T, Strickland D K (1995) LDL receptor-related protein, a multifunctional ApoE receptor, binds secreted beta-amyloid precursor protein and mediates its degradation. Cell 82:331-340.
Li Q, Sudhof T C (2004) Cleavage of amyloid-beta precursor protein and amyloid-beta precursor-like protein by BACE 1. J Biol Chem 279:10542-10550.
Li Y, Cam J, Bu G (2001) Low-density lipoprotein receptor family: endocytosis and signal transduction. Mol Neurobiol 23:53-67.
Lichtenthaler S F, Dominguez D I, Westmeyer G G, Reiss K, Haass C, Saftig P, De Strooper B, Seed B (2003) The cell adhesion protein P-selectin glycoprotein ligand-1 is a substrate for the aspartyl protease BACE1. J Biol Chem 278:48713-48719.
Liscum L, Arnio E, Anthony M, Howley A, Sturley S L, Agler M (2002) Identification of a pharmaceutical compound that partially corrects the Niemann-Pick C phenotype in cultured cells. J Lipid Res 43:1708-1717.
May P, Reddy Y K, Herz J (2002) Proteolytic processing of low density lipoprotein receptor-related protein mediates regulated release of its intracellular domain. J Biol Chem 277:18736-18743.
Mikhailenko I, Battey F D, Migliorini M, Ruiz J F, Argraves K, Moayeri M, Strickland D K (2001) Recognition of alpha 2-macroglobulin by the low density lipoprotein receptor-related protein requires the cooperation of two ligand binding cluster regions. J Biol Chem 276:39484-39491.
Orlando L R, Dunah A W, Standaert D G, Young A B (2002) Tyrosine phosphorylation of the metabotropic glutamate receptor mGluR5 in striatal neurons. Neuropharmacology 43:161-173.
Pietrzik C U, Busse T, Merriam D E, Weggen S, Koo E H (2002) The cytoplasmic domain of the LDL receptor-related protein regulates multiple steps in APP processing. Embo J 21:5691-5700.
Pietrzik C U, Yoon I S, Jaeger S, Busse T, Weggen S, Koo E H (2004) FE65 constitutes the functional link between the low-density lipoprotein receptor-related protein and the amyloid precursor protein. J Neurosci 24:4259-4265.
Quinn K A, Pye V J, Dai Y P, Chesterman C N, Owensby D A (1999) Characterization of the soluble form of the low density lipoprotein receptor-related protein (LRP). Exp Cell Res 251:433-441.
Rebeck G W, Moir R D, Mui S, Strickland D K, Tanzi R E, Hyman B T (2001) Association of membrane-bound amyloid precursor protein APP with the apolipoprotein E receptor LRP. Brain Res Mol Brain Res 87:238-245.
Riddell D R, Christie G, Hussain I, Dingwall C (2001) Compartmentalization of betasecretase (Asp2) into low-buoyant density, noncaveolar lipid rafts. Curr Biol 11: 1288-1293.
Rozanov D V, Hahn-Dantona E, Strickland D K, Strongin A Y (2004) The low density lipoprotein receptor-related protein LRP is regulated by membrane type-1 matrix metalloproteinase (MT1-MMP) proteolysis in malignant cells. J Biol Chem 279:4260-4268.
Sever S, Damke H, Schmid S L (2000) Dynamin:GTP controls the formation of constricted coated pits, the rate limiting step in clathrin-mediated endocytosis. J Cell Biol 150:1137-1148.
Sinha S, Anderson J P, Barbour R, Basi G S, Caccavello R, Davis D, Doan M, Dovey H F, Frigon N, Hong J, Jacobson-Croak K, Jewett N, Keim P, Knops J, Lieberburg I, Power M, Tan H, Tatsuno G, Tung J, Schenk D, Seubert P, Suomensaari S M, Wang S, Walker D, John V, et al. (1999) Purification and cloning of amyloid precursor protein beta-secretase from human brain. Nature 402:537-540. Trommsdorff M, Borg J P, Margolis B, Herz J (1998) Interaction of cytosolic adaptor proteins with neuronal apolipoprotein E receptors and the amyloid precursor protein. J Biol Chem 273:33556-33560.
Trommsdorff M, Gotthardt M, Hiesberger T, Shelton J, Stockinger W, Nimpf J, Hammer R E, Richardson J A, Herz J (1999) Reeler/Disabled-like disruption of neuronal migration in knockout mice lacking the VLDL receptor and ApoE receptor 2. Cell 97:689-701.
Ulery P G, Beers J, Mikhailenko I, Tanzi R E, Rebeck G W, Hyman B T, Strickland D K (2000) Modulation of beta-amyloid precursor protein processing by the low density lipoprotein receptor-related protein (LRP). Evidence that LRP contributes to the pathogenesis of Alzheimer's disease. J Biol Chem 275:7410-7415.
Vassar R, Bennett B D, Babu-Khan S, Kahn S, Mendiaz E A, Denis P, Teplow D B, Ross S, Amarante P, Loeloff R, Luo Y, Fisher S, Fuller J, Edenson S, Lile J, Jarosinski M A, Biere A L, Curran E, Burgess T, Louis J C, Collins F, Treanor J, Rogers G, Citron M (1999) Beta-secretase cleavage of Alzheimer's amyloid precursor protein by the transmembrane aspartic protease BACE. Science 286:735-741.
von Arnim C A, Tangredi M M, Peltan I D, Lee B M, Irizarry M C, Kinoshita A, Hyman B T (2004) Demonstration of BACE ({beta}-secretase) phosphorylation and its interaction with GGA1 in cells by fluorescence-lifetime imaging microscopy. J Cell Sci 117:5437-5445.
Walter J, Fluhrer R, Hartung B, Willem M, Kaether C, Capell A, Lammich S, Multhaup G, Haass C (2001) Phosphorylation regulates intracellular trafficking of betasecretase. J Biol Chem 276:14634-14641.
Yan R, Bienkowski M J, Shuck M E, Miao H, Tory M C, Pauley A M, Brashier J R, Stratman N C, Mathews W R, Buhl A E, Carter D B, Tomasselli A G, Parodi L A, Heinrikson R L, Gurney M E (1999) Membrane-anchored aspartyl protease with Alzheimer's disease beta-secretase activity. Nature 402:533-537.
   Equivalents

All references disclosed herein are incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for modulating β-secretase activity in a subject comprising contacting a mammalian cell with an agent that reduces the amount or rate of binding of β-secretase (BACE) with the low density lipoprotein receptor-related protein (LRP).

2. The method according to claim 1, wherein the agent is an agent which binds to the BACE protein.

3-5. (canceled)

6. The method according to claim 1, wherein the agent is an agent that binds to LRP.

7-9. (canceled)

10. The method according to claim 1, wherein said contacting occurs in vitro.

11. A method for treating or preventing Alzheimer's disease, comprising administering to an animal one or more agents that bind to the BACE-binding site on LRP (Group I agents) and/or one or more agents that bind to the LRP-binding site found on BACE (Group II agents) in an amount effective to reduce the rate of BACE binding to LRP.

12. The method according to claim 11, wherein the agent is an agent which binds to the BACE protein.

13-15. (canceled)

16. The method according to claim 11, wherein the agent is an agent that binds to LRP.

17-19. (canceled)

20. The method according to claim 11, wherein the animal is a human.

21. A pharmaceutical composition comprising
one or more agents that bind to the BACE-binding site of LRP (Group I agents) and/or one or more agents that bind to the LRP-binding site of BACE (Group II agents), and
a pharmaceutically acceptable carrier.

22. The pharmaceutical composition according to claim 21, wherein the agent that binds to the BACE-binding site of LRP is an antibody or an antigen binding fragment thereof that binds to the BACE-binding site of LRP.

23. The pharmaceutical composition according to claim 21, wherein the agent that binds to the BACE-binding site on LRP is a functional derivative or fragment of BACE that binds to LRP.

24. The pharmaceutical composition according to claim 21, wherein the agent that binds to the LRP-binding site of BACE is an antibody or an antigen binding fragment thereof that binds to the LRP-binding site of BACE.

25. The pharmaceutical composition according to claim 21, wherein the agent that binds to the LRP-binding site on BACE is a functional derivative or fragment of LRP that comprises a NPxY (SEQ ID NO:3) motif.

26. The pharmaceutical composition according to claim 21, wherein the agent that binds to the LRP-binding site on BACE is a functional derivative or fragment of LRP that comprises an intracellular domain of LRP.

27. A method for identifying compounds that modulate the interaction of LRP and BACE, comprising
providing a reaction mixture that comprises LRP protein and/or a fragment thereof that includes the cytoplasmic tail including the first NPxY (SEQ ID NO:3) motif or an intracellular domain of LRP, and BACE protein or a fragment thereof that binds specifically to LRP,
contacting the reaction mixture with a test compound,
determining a level of interaction of LRP or fragment thereof with BACE in the absence and in the presence of the test compound, and
comparing the level of interaction of LRP or fragment thereof with BACE in the absence and in the presence of the test compound, wherein a test compound that modulates the interaction relative to the level of interaction in the absence of the test compound is a compound that modulates the interaction of LRP with BACE.

28-31. (canceled)

32. A method for identifying compounds that modulate the cleavage of APP by BACE, comprising
providing a reaction mixture that comprises LRP protein and/or a fragment thereof that includes the cytoplasmic tail including the first NPXY (SEQ ID NO:3) motif or an intracellular domain of LRP, BACE protein, and APP,
contacting the reaction mixture with a test compound,
determining a level of cleavage of APP by BACE in the absence and in the presence of the test compound, and
comparing the cleavage in the absence and in the presence of the test compound, wherein a test compound that modulates cleavage of APP relative to the level of cleavage in the absence of the test compound is a compound that modulates APP cleavage.

33-36. (canceled)

37. A method for modulating LRP signaling activity in a subject comprising
contacting a mammalian cell with an agent that modulates the amount or rate of binding of β-secretase (BACE) with the low density lipoprotein receptor-related protein (LRP), thereby modulating the cleavage of LRP by BACE.

38. The method according to claim 37, wherein the agent is an agent which binds to the BACE protein.

39-41. (canceled)

42. The method according to claim 37, wherein the agent is an agent that binds to LRP.

43-45. (canceled)

46. The method according to claim 37, wherein said contacting occurs in vitro.


SEQUENCE LISTING (4 sequences)

SEQ ID NO: 1 - DNA, 23 bp, Homo sapiens
agccaccagcaccaccagacttg

SEQ ID NO: 2 - DNA, 24 bp, Homo sapiens
actggttggtaacctcacccatta

SEQ ID NO: 3 - protein, 4 aa, Homo sapiens
  MISC_FEATURE (3)..(3): X is any amino acid
NPXY

SEQ ID NO: 4 - protein, 4 aa, Homo sapiens
  MISC_FEATURE (3)..(3): X is any amino acid
APXA